(19) United States
(12) Patent Application Publication
(10) Pub. No.: US 2011/0129485 A1
(43) Pub. Date: Jun. 2, 2011

(54) Modified Galectin-2 and Uses Thereof

(75) Inventors: Hui Wang, Queensland (AU); Anton P.J. Middelberg, Queensland (AU); Lizhong He, Queensland (AU)

(73) Assignee: The University of Queensland, St. Lucia, Queensland (AU)

(21) Appl. No.: 12/992,662

(22) PCT Filed: May 18, 2009
(86) PCT No.: PCT/AU09/00621

§ 371 Date: Feb. 8, 2011

(30) Foreign Application Priority Data
May 19, 2008 (AU) 2008902459

(51) Int. Cl. A61K 39/00; C07K 14/00; C07H 21/00; C07H 21/04; C12N 15/63; C07K 16/18; A61K 31/7088; A61K 38/16; A61P 37/02; A61K 31/711

(52) U.S. Cl. 424/184.1; 530/350; 536/23.5; 435/320.1; 435/325; 530/389.1; 514/44 R; 514/21.2

(57) ABSTRACT

An isolated modified galectin-2 protein comprising a mutation and/or modification which improves one or more properties of said isolated modified galectin-2 are provided. More particularly, the mutation of galectin-2 is substitution of cysteine 57, preferably with a methionine residue. Modification of an isolated galectin-2 includes a modification of cysteine 75. Modification includes chemical modification by PEGylation or alkylation. Also provided are isolated nucleic acid, genetic constructs comprising said isolated nucleic acids, antibodies, compositions and methods of modulating an immune response that may be useful in therapeutic and/or prophylactic treatment of disease, disorders or considers which involve an immune response is mediated by one or more cytokines or other soluble immunomodulators and/or the immune response is mediated by one or more cells of the immune system.

### FIELD OF THE INVENTION

THIS invention relates to a galectin protein. More particularly, this invention relates to a modified galectin-2 protein with an amino acid substitution and/or modification that improves pharmacological, physicochemical and/or biochemical characteristics. The invention also relates to pharmaceutical compositions comprising the modified galectin-2 and methods of therapy using the modified galectin-2 protein.

### BACKGROUND OF THE INVENTION

Galectin-2 (Gal2) is a noncovalent homodimer, sharing 43% amino acid sequence identity with galectin-1 (1). However galectin-2 shows a distinct character in its expression profile, and its expression seems to be confined to the gastrointestinal tract (2). Gal2 is also known as beta galactoside binding lectin, Lectin I 14, LGALS2 or GAL2. Ozaki et al. (3) reported that galectin-2 regulates secretion of the cytokine lymphotoxin-α and thus affects the degree of inflammation. Also, galectin-2 can induce T cell apoptosis via β-galactoside-specific binding (4). A murine model showed that galectin-2 can down-regulate intestinal inflammation and effect a reduction in intestinal injury and inflammation (5).

One well-known property of galectins is the requirement for a reducing reagent (e.g. β-mercaptoethanol, or dithiothreitol (DTT)) to maintain carbohydrate binding ability. The role of the reducing agent is possibly to protect galectins against random formation of disulfide bonds which may destroy their native structure (6). However, Gal2 is prone to aggregation in the absence of reducing agents, which is problematic for use as a biopharmaceutical agent.

A further issue for pharmaceutical applications of galectin-2 is how to overcome rapid kidney clearance thus improving in vivo circulation half-life and yet retain the biological activity of galectin-2.

### SUMMARY OF THE INVENTION

Galectin-2 represents a potentially valuable clinical agent for treatment and/or prophylaxis of a number of immune-related diseases. It is particularly advantageous to have a galectin-2 that can tolerate systemic exposure during treatment by enhancing the circulating half-life. Therefore the availability of alternative strategies for production of a galectin-2 protein which is stable and is suitable for use as a therapeutic agent is highly desirable. A further desirable property is a galectin-2 protein which is substantially-free from unwanted heterogeneous modification products.

In one broad form, the present invention provides an isolated modified galectin-2 that is particularly amenable for use in therapeutic and/or pharmaceutical applications.

In a first aspect, the present invention provides an isolated modified galectin-2 protein selected from the group consisting of an isolated galectin-2 protein comprising a mutation of cysteine 57 or an analogous residue, an isolated galectin-2 protein comprising a modification of cysteine 75 or an analogous residue and an isolated galectin-2 protein comprising a mutation of cysteine 57 and a modification of cysteine 75 or analogous residues, with respect to a wild-type galectin-2 amino acid sequence.

Preferably, the isolated modified galectin-2 protein has one or more improved properties selected from the group consisting of a physicochemical property, a pharmacodynamic property, a pharmacokinetic property and a biochemical property relative to a wild-type galectin-2 protein and/or a galectin-2 protein which has not undergone the modification. More preferably, the isolated modified galectin-2 has improved properties selected from solubility, stability, reduced immunogenicity, reduced antigenicity, reduced toxicity, in vivo circulation half-life and renal clearance.

In preferred embodiments, the mutation improves solubility and/or stability of said isolated modified galectin-2 protein relative to a wild-type galectin-2 protein.

Preferably, the mutation is a substitution of cysteine 57 with respect to a wild-type galectin-2 amino acid sequence. More preferably, the substitution is a conservative substitution or a non-conservative substitution. Even more preferably, the substitution is a non-conservative substitution

In a preferred embodiment, the cysteine 57 is substituted with an amino acid residue selected from the group consisting of a methionine residue, an alanine residue and a serine residue.

More preferably, the cysteine 57 is substituted with a methionine residue.

In a particularly preferred embodiment, the isolated modified galectin-2 protein comprising a mutation as hereinbefore described has the amino acid sequences as set forth in SEQ ID NO: 3.

Preferably, the modification of an isolated modified protein at cysteine 75 improves pharmacodynamic and/or pharmacokinetic properties of said isolated modified protein relative to a galectin-2 protein which has not been modified at cysteine 75.

In preferred embodiments, modification at cysteine 75 is chemical modification. More preferably, chemical modification is selected from treatment with an alkylating agent and attachment of one or more polyethylene glycol (PEG) molecules. Even more preferably, the chemical modification is attachment of one or more PEG molecules.

Preferably, the or each PEG molecule has an average molecular weight of between about 1000 Da and about 100000 Da.

More preferably, the or each PEG molecule has an average molecular weight of between about 4500 Da and about 70000 Da.

Even more preferably, the or each PEG molecule has an average molecular weight of about 5500 Da.

In preferred embodiments, the one or more PEG molecules is selected from the group consisting of a maleimide PEG, an alkylamide PEG, an iodoacetamide PEG, a p-nitro thio-phenyl PEG, a vinyl sulfone PEG, a mixed disulphide PEG and an ortho-pyridyl-disulphide PEG. More preferably, the one or more PEG molecules is a maleimide PEG.

In a second aspect, the invention provides an isolated nucleic acid which encodes an isolated modified galectin-2 protein of the first aspect.

Suitably, although not limited thereto, said isolated nucleic acid is DNA.

In a preferred embodiment, the isolated nucleic acid comprises a nucleotide sequence as set forth in SEQ ID NO: 5.

In a third aspect, the invention provides a genetic construct comprising an isolated nucleic acid of the second aspect operably-linked to one or more regulatory sequences in a vector.

In one preferred embodiment, the genetic construct is an expression construct. More preferably, the expression construct is suitable for expression of a recombinant protein.

In a fourth aspect, the invention provides a host cell comprising a genetic construct of the fourth aspect.

In one preferred embodiment, the host cell is of prokaryotic origin.

In a fifth aspect, the invention provides an antibody which binds an isolated modified galectin-2 protein of the first aspect, wherein said antibody does not bind a wild-type galectin-2 protein or binds to a wild-type galectin-2 protein with relatively lower affinity.

In a sixth aspect, the invention provides a method of producing an isolated modified galectin-2 protein for use in a pharmaceutical composition, said method including the steps of:
(i) mutating an isolated galectin-2 protein at cysteine 57 or an analogous residue, with respect to a wild-type galectin-2 amino acid sequence; and/or
(ii) modifying an isolated galectin-2 protein at cysteine 75 or an analogous residue, with respect to a wild-type galectin-2 amino acid sequence; or
(iii) modifying the mutated galectin-2 protein of step (i), at cysteine 75 or an analogous residue, with respect to a wild-type galectin-2 amino acid sequence;
   to thereby produce said isolated modified galectin-2 protein for use in a pharmaceutical composition.

In a seventh aspect, the invention provides an isolated modified galectin-2 protein produced according to a method of the sixth aspect.

Preferably, the wild-type galectin-2 amino acid sequence is an amino acid sequence selected from the group consisting of SEQ ID NO: 1 and SEQ ID NO: 2.

Suitably, the isolated modified galectin-2 protein is of human origin.

In an eighth aspect, the invention provides a pharmaceutical composition comprising an isolated modified protein, an isolated nucleic acid or a genetic construct according to any one of the aforementioned aspects, together with a pharmaceutically acceptable carrier, diluent or excipient.

Compositions according to this aspect may be used either prophylactically or therapeutically.

In a ninth aspect, the invention provides a method of modulating an immune response in an animal, said method including the step of administering an effective amount of a pharmaceutical composition of the eighth aspect to thereby modulate the immune response in the animal.

Suitably, the immune response is an inflammatory immune response.

In one preferred embodiment, the immune response is mediated by one or more cytokines or other soluble immunomodulators.

Preferably, the cytokine is lymphotoxin alpha. According to this embodiment, the method modulates an immune response in myocardial infarction, coronary heart disease, and coronary artery disease.

In another preferred embodiment, the immune response is mediated by one or more cells of the immune system.

Preferably, the one or more cells of the immune system are activated T cells.

In preferred embodiments, the method of ninth aspect modulates an immune response in a disease, disorder or condition responsive to inhibition or suppression of T cell activation.

In a tenth aspect, the invention provides a method of treating an animal said method including the step of administering an effective amount of a pharmaceutical composition according to any one of aforementioned aspects to said animal to thereby modulate an immune response in said animal to prophylactically and/or therapeutically treat an inflammatory disease or a disease, disorder or condition responsive to inhibition or suppression of T cell activation.

In preferred embodiments, the inflammatory disease is selected from the group consisting of myocardial infarction, coronary heart disease, and coronary artery disease.

In preferred embodiments of the ninth and tenth aspect, the disease, disorder or condition responsive to inhibition or suppression of T cell activation is selected from the group consisting of inflammatory bowel disease, graft-versus-host disease and an allergic reaction.

An animal can be selected from the group consisting of humans, domestic livestock, laboratory animals, performance animals, companion animals, poultry and other animals of commercial importance, although without limitation thereto.

Preferably, the animal is a mammal.

More preferably, the animal is a human.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1 Ribbon diagram of the dimeric hGal2-lactose complex. The lactose moiety in each monomer is shown in bold stick representation. This figure was produced from the X-Ray crystal-structure of hGal2 (PDB entry 1HLC) (17) using PyMOL.

FIG. 2 Schematic diagram of the multilayer architecture presenting asialofetuin (ASF) for hGal2 binding studies by surface-plasmon resonance (SPR). This architecture was composed with four layers (thiol-SAM/biotin/streptavidin (SA)/ASF) which specifically interact with hGal2 while minimizing non-specific BSA adsorption.

FIG. 3 Expression analysis of human galectin-2 wild type and mutants. L: protein marker; T: total expression; S: soluble expression; I: insoluble expression. Samples of hGal2 WT and C57M were loaded on one Protein 80 Plus LabChip®. Samples of hGal2 C57S and C57A were loaded on another chip.

FIG. 4 Purification and characterisation of hGal2 C57M. (A) Affinity purification. 15 mL of sample was loaded on a 5 mL Lactose-agarose column pre-equilibrated with Buffer PA, followed by two-step elution with lactose-containing buffer PB. Purified hGal2 was eluted with 33% PB buffer. (B) Bioanalyzer® 2100 electropherogram for purified hGal2 C57M.

FIG. 5 LC-MS analysis of hGal2 wild type (theoretical MW 144853 Da) and C57M mutant (theoretical MW 14511 Da).

FIG. 6 Analysis of protein stability using gel-filtration chromatography (Superdex S200). All purified samples were incubated at 4° C. for 3 weeks. (A) hGal2 WT; (B) hGal2 C57M.

FIG. 7 Purification of PEGylated hGal2 C57M using ion-exchange chromatography. A step then gradient elution was applied to a column pre-equilibrated with Buffer IA. Purified PEGylated hGal2 C57M was eluted in the first elution step with 8% Buffer IB.

FIG. 8 PEGylation analysis of hGal2 C57M. (A) Gel-filtration chromatograph (Superdex 200) analysis of hGal2 before and after PEGylation. (B) Bioanalyzer® 2100 analysis of hGal2 before and after PEGylation. L: protein marker; 1: hGal2 C57M; 2: PEGylation product. (C) MALDI-MS analysis of PEGylated hGal2 C57M. Peaks corresponding to mono-PEGylated hGal2 C57M: peak 5 (at mass 20045, single-charged ion), peak 3 (at mass 9953, double-charged ion), and peak 1 (at mass 6634, triple-changed ion). Peaks corresponding to free hGal2 C57M: peak 4 (at mass 14398, single-charged ion) and peak 2 (at mass 7151, double-charged ion).

FIG. 9 Far-UV CD spectra for hGal2 WT, hGal2 C57M, and PEGylated hGal2 C57M.

FIG. 10 Stability analysis of PEGylated hGal2 C57M using gel-filtration chromatography (Superdex S200). Samples were incubated at 4° C. for 3 weeks.

FIG. 11 Kinetic curves of protein adsorption to an ASF-immobilized surface using surface plasmon resonance at a fixed angle (ca. 60°, giving an initial reflectivity about 30%). The system was equilibrated with PBS (20 mM sodium phosphate, 100 mM NaCl, pH 7.2). Loading of (A) BSA (as control); (B) hGal2 WT; (C) hGal2 C57M; (D) PEGylated hGal2 C57M.

FIG. 12 Amino acid sequence alignment of hGal2 (Swiss-Prot ID: P05162; SEQ ID NO: 1), hGal2 WT (SEQ ID NO: 2) and hGal2 C57M (SEQ ID NO: 3).

FIG. 13 DNA sequence alignment of hGal2 WT (wild-type; SEQ ID NO: 4) and hGal2 C57M (SEQ ID NO: 5).

FIG. 14 Sequence Listing in Patent-In 3.3 format.

### DETAILED DESCRIPTION OF THE INVENTION

The present invention arises, at least partly, from the inventors' elucidation of amino acid residues in human galectin-2 (hGal2) that are particularly amenable to mutation and/or modification. Moreover, manipulation of hGal2 at these residues results in a galectin-2 protein which is particularly suitable for use as a biopharmaceutical agent since the galectin-2 protein of the present invention displays improved physicochemical, pharmacological (such as pharmacokinetic and pharmacodynamic) and biochemical properties. A further advantage conferred by an approach of the present invention is production of a stable and soluble galectin-2 mutant which may, potentially, be chemically modified in a homogeneous fashion. Furthermore, a modified galectin-2 protein of the present invention is well-suited for large scale manufacturing processes, such as are desired for commercial pharmaceutical preparations.

Therefore in one broad form, the invention relates to an isolated modified galectin-2 protein which has been chemically modified and mutated to thereby produce an isolated modified galectin-2 protein with preferably enhanced stability, solubility, reduced immunogenicity, reduced antigenicity and/or reduced toxicity and potentially improved therapeutic applications. In preferred embodiments, the isolated modified galectin-2 protein is site-specifically chemically modified.

Although not wishing to be bound by any particular theory, the galectins are a family of animal lectins, which are widely distributed from lower to higher vertebrates. At the time of writing, the galectin family of proteins are defined by two criteria: affinity for β-galactosides and significant sequence similarity in the carbohydrate recognition domain (CRD) of about 130 amino acids. Galectins function both extracellularly and intracellularly. By binding to glycoconjugates (glycoproteins and glycolipids) at the cell surface, or in the extracellular matrix, galectins can mediate cell-cell and cell-extracellular matrix adhesion, and modulate processes including mitosis, apoptosis and cell-cycle progression. Intracellularly, they can shuttle between the nucleus and cytoplasm and interact with intracellular proteins, thereby engaging in cellular functions such as pre-mRNA splicing and the regulation of cell growth.

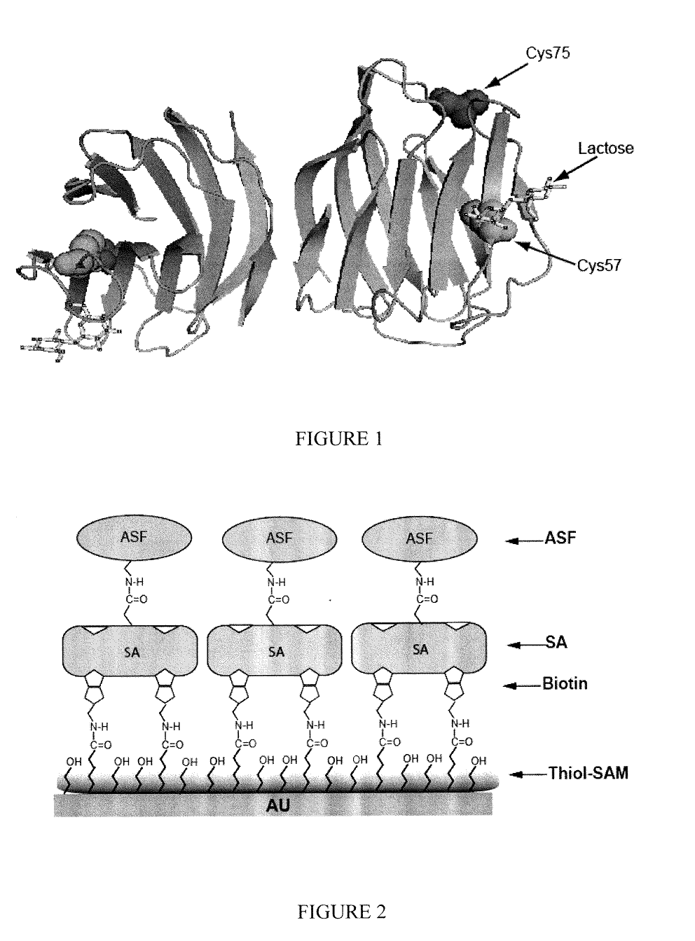

Human galectin-2 (hereinafter referred to as ‘hGal2’) comprises two cysteines, namely cysteine at position 57 (Cys57) and cysteine at position 75 (Cys75), or analogous residues. Reference is made to FIG. 1, which shows the three-dimensional structure of the hGal2-lactose complex. Cys75 is distant from the carbohydrate recognition domain (CRD) of hGal2, whereas Cys57 is within the CRD region.

The isolated proteins of the present invention utilise Cys57 (or an analogous residue) as the mutation site whilst leaving Cys75 (or an analogous residue) as non-mutated yet modified. A rationale behind this selection is that since Cys75 is distant from the CRD domain (ie. the ligand binding domain) this enables modification of hGal2 at Cys75 without a potential significant disturbance of the CRD (and consequently biological) activity of galectin-2.

While the present invention has been primarily exemplified using human galectin-2 protein, it will be appreciated that in other forms, the invention also extends to any other eukaryotic or mammalian ortholog of galectin-2 protein inclusive of orthologs of mammals of commercial significance such as horses, cows, camels, goats, pigs and sheep and also companion mammals such as dogs and cats. Any variant of galectin-2 protein can be used in the present invention provided it retains a suitable level of galectin-2 activity.

“Variants” of galectin-2 include within their scope naturally-occurring variants such as allelic variants, orthologs and homologs and artificially created mutants, for example.

It will also be appreciated by a skilled addressee that galectin-2 has a number of synonyms and at the time of writing, is also known in the art as beta galactoside binding lectin, Lectin I 14, LGALS2 or GAL2. “Galectin-2” and “Gal2” will be used be interchangeably herein.

### Isolated Proteins and Isolated Nucleic Acids

In light of the foregoing, it will be appreciated that in particularly broad aspects, the present invention provides an isolated modified galectin-2 protein and in preferred aspects, an isolated mutant galectin-2 protein and/or an isolated derivative galectin-2 protein. In other preferred aspects, the invention provides an isolated modified galectin-2 protein comprising a mutation and a modification. It will be appreciated that modified galectin-2 proteins of the present invention can be prepared to increase serum half-life, reduce undesired or adverse immune and toxic responses against galectin-2 protein and/or facilitate purification or preparation by improving stability and/or solubility.

It will be appreciated that said mutation and/or modification of galectin-2 as described herein typically occurs without a substantial loss-of-function or biological activity to the isolated modified galectin-2 protein compared to wild-type galectin-2 protein or an unmodified galectin-2 protein. In general preferred embodiments, the isolated modified galectin-2 protein retains a sufficient level of biological activity so that said modified galectin-2 protein is able function as an effective biopharmaceutical as required.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids or chemically-derivatized amino acids as are well understood in the art.

A “peptide” is a protein having less than fifty (50) amino acids.

A “polypeptide” is a protein having fifty (50) or more amino acids.

Proteins and peptides may be useful in native, chemical synthetic or recombinant synthetic form.

As used herein, by “synthetic” is meant not naturally occurring but made through human technical intervention. In the context of synthetic proteins and nucleic acids, this encompasses molecules produced by recombinant or chemical synthetic and combinatorial techniques as are well understood in the art.

The terms “mutant”, “mutation” and “mutated” are used herein to preferably encompass amino acid substitutions introduced into a galectin-2 protein or a fragment thereof, that generally improve physicochemical and/or biochemical properties of the galectin-2 protein. In particularly preferred embodiments, said mutations generally improve stability and/solubility of a galectin-2 protein when compared to wild-type galectin-2 protein. In other preferred embodiments, a mutation of the present invention improves stability and/or solubility against in vitro aggregation. A mutation of the present invention may also generally preserve the tertiary structure of galectin-2. A mutation may be a conservative substitution or non-conservative substitution. A further beneficial effect of mutation of galectin-2 is that it obviates or substantially removes or alleviates the requirement of a reducing agent (such as DTT but not limited thereto) which is typically present prevent aggregation of galectin-2 during storage. It will also be appreciated that in certain other preferred embodiments, a suitable mutation is one in which the solubility of galectin-2 is retained or improved, especially in the context of in vitro or in vivo recombinant expression.

In preferred embodiments, galectin-2 is mutated at a cysteine residue located at position 57, or an analogous residue, according to numbering of a wild-type galectin-2 amino acid sequence. In particularly preferred embodiments, the wild-type galectin-2 amino acid sequence is an amino acid sequence selected from the group consisting of SEQ ID NO:1 and SEQ ID NO:2.

It is well understood in the art that some amino acids may be changed to others with broadly similar properties without changing the nature of the activity of the protein or the structure of the protein (conservative substitutions).

Generally, non-conservative substitutions which are likely to produce the greatest changes in protein structure and function are those in which (a) a hydrophilic residue (e.g. Ser or Thr) is substituted for, or by, a hydrophobic residue (e.g. Ala, Leu, Ile, Phe or Val); (b) a cysteine or proline is substituted for, or by, any other residue; (c) a residue having an electropositive side chain (e.g. Arg, His or Lys) is substituted for, or by, an electronegative residue (e.g. Glu or Asp) or (d) a residue having a bulky hydrophobic or aromatic side chain (e.g. Val, Ile, Phe or Trp) is substituted for, or by, one having a smaller side chain (e.g. Ala, Ser) or no side chain (e.g. Gly).

It will be appreciated by a skilled addressee that in the context of the present invention, suitability of the substitution on protein structure, stability, solubility and/or function is not, prima facie, readily predictable. By way of example, Cys57→Met substitution results in a highly soluble form of galectin-2.

In particularly preferred embodiments of the present invention, Cys57 of galectin-2 is mutated to a methionine residue (hereinafter referred to as C57M).

A person of skill in the art would readily appreciate that galectin-2 mutants can be created by mutagenizing a protein or alternatively, by mutagenizing an isolated nucleic acid encoding a protein, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra, chemical modification of proteins by hydroxylamine (18), incorporation of dNTP analogs into nucleic acids (19), PCR-based random mutagenesis such as described in Stemmer, 1994, Proc. Natl. Acad. Sci. USA 91 10747 or Shafikhani et al., 1997, Biotechniques 23 304, or mutagenesis kits such as Diversity™ and QuickChange™ are also contemplated by way of example.

A non-limiting example of the preparation of mutant galectin-2 by site-directed nucleic acid mutagenesis, and in particular C57M, is provided in the Materials and Methods section of Example 1 contained herein.

The term “nucleic acid” as used herein designates single- or double-stranded mRNA, RNA, cRNA and DNA inclusive of cDNA, genomic DNA and DNA-RNA hybrids. Nucleic acids may also be conjugated with fluorochromes, enzymes and peptides as are well known in the art.

In general preferred embodiments, an isolated modified galectin-2 protein comprising a mutation and/or modification of the present invention improves one or more properties of said modified galectin-2 protein.

As used herein, by “improves”, “improved”, “improve”, “improvement” or “improving” is meant a mutation, substitution and/or modification incorporated or introduced into a galectin-2 protein (as described herein) that has a beneficial, higher, better, increased, enhanced or otherwise superior effect on one or more properties of said modified and/or mutated galectin-2 protein when compared to or relative to a galectin-2 protein which has not been modified and/or mutated or a wild-type galectin-2 protein. Said one or more properties are selected from the group consisting of a physicochemical property, a biochemical property, a pharmacokinetic property and a pharmacodynamic property. An improvement may be quantified as a 5%, 10%, 15%, 20%, 25%, 30%, 35%, 40%, 45%, 50%, 55%, 60%, 65%, 70%, 75%, 80%, 85%, 90%, 95% or 100% or more increase in said one or more properties as measured, assayed, determined or otherwise compared to against either a wild-type galectin-2 protein or a galectin-2 protein which has not been modified and/or mutated.

In certain preferred embodiments, an improvement is in relation to protein stability which may manifest as an ability to withstand aggregation in the absence of reducing agents for a prolonged period of time. In other preferred embodiments, protein solubility may be improved as evidenced by an increase in expression of a soluble form of galectin-2 in recombinant expression systems. Protein solubility may also relate to in vivo solubility.

The term “pharmacokinetic” as used herein broadly refers to what the body does to a drug and more particularly, to the kinetics of drug liberation, absorption, distribution and elimination (ie. metabolism and excretion) and includes serum half-life of a drug and renal clearance rate.

The term “pharmacodynamic” as used herein broadly refers to what a drug does to the body and more particularly, refers to the relationship between drug concentration at the site of action(s) and the biochemical and pharmacological response and is inclusive of drug toxicity, immunogenicity and antigenicity.

In other particular aspects, the present invention provides an isolated galectin-2 protein which can be considered a derivative of galectin-2 which is modified and in particularly preferred embodiments, chemically modified as described herein.

It is preferable that a galectin-2 protein is site-specifically modified in a manner that does not result in significant alteration or perturbation of galectin-2 secondary structure and also preferably allows galectin-2 to homodimerise. Advantageously, although not exclusively, modification may result in therapeutic benefits such as longer in vivo circulation life time, decreased toxicity and immunogenicity, increased solubility and facilitation of purification and preparation.

In preferred embodiments, a galectin-2 modified or derivative protein is modified at Cys75 or an analogous residue. Cys75 represents a particularly amenable site for modification since addition of a moiety to Cys75 minimises, and preferably eliminates, allosteric modification of CRD activity and other problems arising from steric hindrance close to the ligand binding domain. In particularly preferred embodiments, a galectin-2 protein is chemically modified at Cys75.

It will be appreciated by a skilled addressee that is desirable, and indeed advantageous, to produce a homogenous modified therapeutic agent or a single positional isomer of a therapeutic drug target for a variety of reasons, including better consistency and control over the processes for producing the agent, consistent properties allow improved analytical characterisation of the product in vivo and ideally, straightforward regulatory approval.

A particularly preferred chemical moiety for derivatisation of a galectin-2 protein is polyethylene glycol (PEG). Modification by PEGylation may occur at random positions within a protein or a predetermined position and may include one, two, three or more attached PEG molecules as described hereinbelow.

The terms “PEGylated galectin-2” or “PEG-galectin-2” as used herein refer to a galectin-2 comprising one or more linked PEG molecules.

It is noted that a galectin-2 protein comprising an attached PEG molecule may also be known as a conjugated protein whilst a galectin-2 protein lacking an attached PEG may be referred to as an unconjugated protein.

PEGylation may significantly improve the physicochemical properties (solubility and stability) of biopharmaceuticals such as galectin-2 while also increasing in vivo circulation half-life (decreased enzymatic degradation and decreased kidney clearance). Indeed PEG conveys to molecules such as proteins its physicochemical properties and therefore modifies also biodistribution and solubility of protein-based biopharmaceuticals. More particularly, PEG conjugation may mask a protein's surface and increase the molecular size of the protein, thus reducing its renal ultrafiltration, preventing the approach of antibodies or antigen processing cells and reducing degradation by proteolytic enzymes.

There are several methodologies available for protein PEGylation, and conjugation chemistries. Reference is made to Roberts et al (2002) Advanced Drug Delivery Reviews 54: 459-476 and Zalipsky (1995) Advanced Drug Reviews 16: 157-182 which provide non-limiting examples of several methodologies available for protein PEGylation, and conjugation chemistries and are incorporated herein by reference. One of the most common methods is lysine-directed conjugation, as PEG with amine-active groups such as N-hydroxysuccinimide (NHS) can readily react with the ε-amino group of lysine and/or the primary amino group at the N-terminus. This straightforward method is often the first choice for protein PEGylation because most proteins have surface-available lysine residues. However, this random PEGylation approach often results in a mixture of products containing different numbers of PEG molecules conjugated at different sites. The resulting mixed products are usually difficult to purify at preparative scale, complicating detailed characterization and functional analysis of final products thus compromising their biomedical application. Although the FDA has previously approved a mixture of PEGylation isomers based on the evidence that the reaction is reproducible, at the time of writing it required compulsory characterization of each isomer if a mixed product is used (13).

In order to avoid heterogeneous products resulting from random PEGylation, different strategies have been developed for site-specific PEGylation of proteins, by a combination of site-specific mutagenesis and residue-specific chemical reaction. A method is selective thiol PEGylation targeting free cysteine residues. This strategy has been successfully used for site-directed PEGylation of recombinant interleukin-2 as described in Goodson et al, 1990, Bio-Technology 8: 343; therapeutic antibody fragments (20); and recombinant immunotoxins (21). Other methods include the construction of lysine-deficient mutants to enable site-specific mono-PEGylation at the N-terminus (22)), construction of chimeric proteins for transglutaminase-catalyzed PEGylation targeting the substrate glutamine residues (23), and using glycosyltransferases to attach PEG to O-glycans (24).

In preferred embodiments, galectin-2 is site-specifically PEGylated. In particularly preferred embodiments, galectin-2 is site-specifically or selectively PEGylated at Cys75 of galectin-2.

PEG is a well-known water soluble polymer represented by the following general formula:

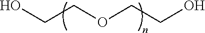

PEG may also have a branched or unbranched structure. Non-limiting examples of branched PEGs suitable for conjugation to galectin-2 are provided in U.S. Pat. No. 5,643,575.

In particularly preferred embodiments, the PEG is a branched structure.

The PEG molecule suited for use in the present invention may be of any molecular weight between about 1 kDa to about 100 kDa, as practically desired. Typically, although not exclusively, PEG preparations exist as a heterogeneous mixture of PEG molecules either above or below the stated molecular weight. By way of example, the PEG may have an average molecular weight of about 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 25000, 30000, 35000, 40000, 45000, 50000, 55000, 60000, 65000, 70000, 75000, 80000, 85000, 90000, 95000 or 100000 Da.

Preferably, the PEG has an average molecular weight of about between about 4500 Da to about 70000 Da.

More preferably, the PEG has an average molecular weight of about 5500 Da.

The choice of molecular weight of PEG in PEGylated galectin-2 is dependent upon a range of factors inclusive of how PEGylated galectin-2 will be used therapeutically, the desired dosage, circulation time, resistance to proteolysis, toxicity and immunogenicity. Reference is made to Katre, 1993, Advanced Drug Delivery Reviews, 10: 91, which provides a review of PEG and its application in enhancing the properties, and in particular therapeutic properties, of proteins. A skilled addressee will appreciate that PEG molecules may be chemically synthesized or are readily available in commercial foul) for example from NOF Corporation (Tokyo, Japan).

There are a number of methods available to attach, link, complex or otherwise conjugate PEG to a galectin-2 protein of the present invention. An important consideration when applying a suitable method is the effect on functional or antigenic domains of the protein. By way of example only, PEG may be conjugated to galectin-2 by way of covalent binding through amino acid residues by a reactive group, such as a free amino, imino or carboxyl group. Sulfhydryl groups may also be employed as the reactive group for PEG conjugation. A further example of PEG attachment to an amino acid residue is selective thiol PEGylation of a cysteine residue by methods known in the art, such as those hereinbefore described. Non-limiting examples of PEG derivatives having thiol-selective end groups include maleimides, vinyl sulfones, iodoactamides and thiols.

It is contemplated that in preferred embodiments, a PEG is selected from the group consisting of a maleimide PEG, an alkylamide PEG, an iodoacetamide PEG, a p-nitro thio-phenyl PEG, a vinyl sulfone PEG, a mixed disulphide PEG and an ortho-pyridyl-disulphide PEG. In particularly preferred embodiments, the PEG is the maleimide PEG. More preferably, the maleimide PEG is maleimide-PEG-5 kDa.

The PEG molecule may be coupled or attached to galectin-2 either directly or by way of a linker. Veronese, 2003, Biomaterials, 22: 405-417 provides an overview of PEG conjugation chemistry and is incorporated herein by reference. Linkerless methods for PEG conjugation employ compounds such as maleimide-PEG and similar compounds which can directly attach PEG to the protein of interest. Non-limiting examples of linkerless methods for coupling PEG to proteins are described in U.S. Pat. Nos. 5,349,052 and 6,646,110; Greenwald et al, Crit Rev Ther Drug Carrier Syst, 2000, 17:101-61, which are incorporated herein by reference.

In linker-dependent systems, non-limiting examples of linkers includes urethane linkers such as described in U.S. Pat. No. 5,612,460 and enzyme-based systems using O-glycans as described in DeFrees et al, 2006, Glycobiology 16: 833-843, which are incorporated herein by reference.

It is contemplated that the degree of PEG substitution of galectin-2 may vary. By way of example, the PEGylated proteins may be linked to 1, 2, 3, 4 or more PEG molecules. In preferred embodiments, each monomer of a galectin-2 homodimer is attached to a single PEG (referred to herein as “monoPEGylated” or “monoPEGylation”) with the following formula: PEG-Gal2-Gal2-PEG. Other PEGylation states are contemplated by the invention such as PEG-Gal2-Gal2 although without limitation thereto.

In the context of the present invention, by “derivative”, “derivatization” or “derivatised” is meant a galectin-2 protein which is altered or modified, for example by attachment, linkage, conjugation or complexing with other chemical moieties or by post-translational modification techniques as would be understood in the art. It will be appreciated that derivatization of galectin-2 as herein described preferably occurs without a substantial loss-of-function or biological activity of a wild-type galectin-2 protein.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride: amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; alkylation with iodoacetate, ethyleneimine or iodoacetaminde; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

In other particularly preferred embodiments. Cys75 of galectin-2 is alkylated. It will be appreciated that alkylation of a cysteine residue can be performed by a number of reagents inclusive of iodoacetic acid, iodoacetamide, 5-I-AEDANS and N-ethylmaleimide but without limitation thereto. Reference is made to Whitney et al, 1986, Biochem J, 238: 683-689, which provides non-limiting examples of alkylation of a galectin protein and is incorporated herein by reference. Alkylation of Cys75 of galectin-2 may also be achieved using standard methods such as those described in Chapter 15 of CURRENT PROTOCOLS IN PROTEIN SCIENCE, Coligan et al. Eds (John Wiley & Sons, 1995-2000).

The invention also contemplates fragments of an isolated modified protein of the invention.

A “fragment” is a segment, domain, portion or region of a galectin-2 protein, which constitutes less than 100% of the galectin-2 protein.

A fragment may comprise at least 5, 10, 20, 30, 40, 50, 60, 70, 80, 90, 100, 110 or 120 contiguous amino acids of a galectin-2 protein, although without limitation thereto.

A peptide may be a fragment, for example a fragment comprising at least 6, 10, 12, 15, 20, 30 and up to 60 contiguous amino acids. Peptide fragments may be obtained through the application of standard recombinant nucleic acid techniques or synthesized using conventional liquid or solid phase synthesis techniques. For example, reference may be made to solution synthesis or solid phase synthesis as described, for example, in Chapter 18 of CURRENT PROTOCOLS IN PROTEIN SCIENCE, Coligan et al. Eds (John Wiley & Sons, 1995-2000). Alternatively, peptides can be produced by digestion of a polypeptide of the invention with proteinases such as endoLys-C, endoArg-C, endoGlu-C and V8-protease. The digested fragments can be purified by chromatographic techniques as are well known in the art.

In particular embodiments, the fragment may be a “biologically active fragment” which displays or retains biological, structural and/or physical activity of a given protein, or an encoding nucleic acid. In certain forms of this embodiment, the biologically active fragment of galectin-2 has the ability to bind a suitable ligand such as, but not limited to, asialofetuin, glycolipid GM1 and β1-integrin. Other methods for biological activity assays may include, but not limited to, binding profile determined by immunohistochemistry, and apoptosis in activated T cells.

In light of the foregoing, it will be appreciated that an isolated galectin-2 protein of the present invention may encompasses a combination of a mutation and a modification as hereinbefore described. In general preferred embodiments, a galectin-2 protein of the invention includes mutation at Cys57 and chemical modification at Cys75.

The invention contemplates monomeric and multimeric forms of the mutant and/or derivative galectin-2 proteins. Typically, although not exclusively, the proteins of the invention are in a form that is comparable to the native state of galectin-2. Preferably, the isolated proteins of the present invention are homodimeric forms of galectin-2.

As described above, the activity of an isolated galectin-2 protein (including either isolated galectin-2 protein being a mutant, a derivative or combination thereof) of the present invention can be ascertained using a galectin-2 assay as is known in the art. Preferably, an isolated galectin-2 protein, or fragment thereof, of the present invention retains at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% activity of the wild-type and/or unconjugated galectin-2 protein.

It may be necessary to ascertain the tertiary structure, stability and/or solubility of the isolated proteins of the present invention. This may be particularly useful for determining a mutation or modification has a detrimental or adverse effect on galectin-2 physicochemical and/or biochemical characterisitics. Various methods may be utilised including, but not limited to, chromatographic methods such as size-exclusion chromatography, physical methods such as circular dichroism and biological assays. Reference is made to the Examples section contained herein which exemplifies some of the aforementioned methods.

It will be appreciated that the present invention provides isolated nucleic acids and genetic constructs comprising the same that in particularly preferred embodiments, facilitate recombinant protein expression. Furthermore, expression from said expression construct may be performed in a prokaryotic or eukaryotic system.

It will be well appreciated by a person of skill in the art that the isolated nucleic acids of the invention ention can be conveniently prepared by a person of skill in the art using standard protocols such as those described in Chapter 2 and Chapter 3 of CURRENT PROTOCOLS IN MOLECULAR BIOLOGY (Eds. Ausubel et al. John Wiley & Sons NY, 2000-2009).

In preferred embodiments, the isolated nucleic acid of the invention is DNA. In particularly preferred embodiments that contemplate a C57M galectin-2 protein, the isolated nucleic acid comprises a nucleotide sequence as set forth in SEQ ID NO:5

The invention also contemplates variant galectin-2 nucleic acids having one or more codon sequences altered by taking advantage of codon sequence redundancy.

A particular example of a variant galectin-2 nucleic acid is optimization of a nucleic acid sequence according to codon usage, as is well known in the art. This can effectively “tailor” a nucleic acid for optimal expression in a particular organism, or cells thereof, where preferential codon usage has been established.

The present invention further contemplates use of modified purines (for example inosine, methlinosine and methyladenosine) and modified pyrimidines (for example, thiouridine and methylcytosine) in nucleic acids of the invention.

In one particular embodiment, an isolated nucleic acid of the present invention is operably linked to one or more regulatory nucleotide sequences in a genetic construct. A person skilled in the art will appreciate that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of the isolated nucleic acid. Typically, although not exclusively, genetic constructs are designed for more than one application. By way of example only, a genetic construct whose intended end use is recombinant protein expression in a eukaryotic system may have incorporated nucleotide sequences for such functions as cloning and propagation in prokaryotes over and above sequences required for expression. An important consideration when designing and preparing such genetic constructs are the required nucleotide sequences for the intended application.

A person skilled in the art will appreciate that the isolated nucleic acid may be inserted into a vector to produce or generate said genetic construct by a variety of recombinant techniques using standard protocols as for example described in Sambrook et al., MOLECULAR CLONING, A Laboratory Manual (Cold Spring Harbor Press, 1989), which is incorporated herein by reference.

In view of the foregoing, it is evident to a person of skill in the art that genetic constructs are versatile tools that can be adapted for any one of a number of purposes.

Therefore in one particular aspect, the invention provides a genetic construct comprising an isolated nucleic acid of the invention operably linked to one or more regulatory nucleotide sequences in a vector.

In particular embodiments, the invention contemplates an expression construct comprising an isolated nucleic acid operably-linked to one or more regulatory nucleotide sequences in an expression vector. It will be appreciated that the aforementioned expression construct is particularly suitable for recombinant protein expression. Preferably, the expression construct comprises at least a promoter and in addition, one or more other regulatory nucleotide sequences which are required for manipulation, propagation and expression of recombinant DNA.

A “vector” or “expression vector” may be either a self-replicating extra-chromosomal vector such as a plasmid, or a vector that integrates into a host genome, inclusive of vectors of viral origin such as adenovirus, lentivirus, poxvirus and flavivirus vectors as are well known in the art.

By “operably linked” is meant that said regulatory nucleotide sequence(s) is/are positioned relative to the recombinant nucleic acid of the invention to initiate, control, regulate or otherwise direct transcription and/or other processes associated with expression of said nucleic acid.

“Regulatory nucleotide sequences” present in the genetic or expression construct may include an enhancer or activator sequences, promoter, splice donor/acceptor signals, Kozak sequence, leader or signal sequences for secretion of a translated protein, ribosomal binding sites, nucleic acid packaging signals, terminator and polyadenylation sequences, as are well known in the art and facilitate expression of the nucleotide sequence(s) to which they are operably linked, or facilitate expression of an encoded protein. Regulatory nucleotide sequences will generally be appropriate for the host cell or organism used for expression. Numerous types of appropriate genetic constructs and suitable regulatory sequences are known in the art for a variety of host cells.

With regard to promoters, constitutive promoters (such as CMV, SV40, vaccinia, HTLV1 and human elongation factor promoters) and inducible/repressible promoters (such as tet-repressible promoters and IPTG-, metallothionine- or ecdysone-inducible promoters) are well known in the art and are contemplated by the invention as are tissue-specific promoters such as α-crystallin promoters. It will also be appreciated that promoters may be hybrid promoters that combine elements of more than one promoter.

Preferably, said promoter is operable in a prokaryotic cell and preferably a bacterial cell. Non-limiting examples include T7 promoter, tac promoter and T5 promoter.

Preferably, said genetic or expression construct also includes one or more selectable markers suitable for the purposes of selection of transformed bacteria (such as bla, kanR and tetR) or transformed eukaryotic cells may be selected by markers such as hygromycin, G418 and puromycin, although without limitation thereto.

Among vectors preferred for use in cells of prokaryotic origin include pQE60 available from Qiagen, pGEX series of vectors available from GE Life Sciences and pET vector system available from Novagen.

Genetic constructs may be introduced into cells or tissues, inclusive of cells capable of recombinant protein production, by any of a number of well known methods typically referred to as “transfection”, “transduction”, “transformation” and the like. Non-limiting examples of such methods include transformation by heat shock, electroporation, DEAE-Dextran transfection, microinjection, liposome-mediated transfection (e.g. lipofectamine, lipofectin), calcium phosphate precipitated transfection, viral transformation, protoplast fusion, microparticle bombardment and the like.

It is readily contemplated that any recombinant protein expression system may be used for the present invention such as bacterial, yeast, plant, mammalian cell lines such as lymphoblastoid cell lines and splenocytes isolated from transformed host organisms such as humans and mice and insect-based expression systems but is not limited thereto. It will be appreciated that the recombinant protein expression system employed may be chosen on the basis of suitability for expression of soluble and stable protein.

In one preferred embodiment, recombinant protein expression occurs in cells of prokaryotic origin. Suitable host cells for recombinant protein expression are bacterial cells such as Escherichia coli (BL21 and various derivative strains thereof which have been optimised for certain applications, such as Rosetta and DE3, for example) and Bacillus subtilis, although without limitation thereto. Preferably the host cell is Escherichia coli BL21 DE3.

In another preferred embodiment, recombinant expression occurs in insect cells which are suited to viral-based recombinant expression e.g. Sf9 cells.

To facilitate recombinant protein purification, a fusion partner sequence may be included with the galectin-2 protein of the present invention. That is, a genetic construct of the present invention may also include a fusion partner (typically provided by a vector or an expression vector) so that the recombinant protein of the invention is expressed as a fusion protein with said fusion partner. The main advantage of fusion partners is that they assist identification and/or purification of said fusion protein. However it will also be appreciated that the choice of fusion partner may also assist with protein properties such as stability, solubility and the like. Non-limiting examples of such proteins include Protein A, glutathione S-transferase (GST), green fluorescent protein (GFP) maltose-binding protein (MBP). hexahistidine (HIS₆) and epitope tags such as V5, FLAG, haemagglutinin and c-myc tags.

The fusion partner sequence facilitates fusion protein binding to an affinity matrix to enable protein purification and/or detection. For the purposes of fusion polypeptide purification by affinity chromatography, relevant matrices for affinity chromatography are antibody, protein A- or G-, glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In many cases, the fusion partner can be cleaved by an appropriate protease or chemical reagent to release the galectin-2 protein from the fusion partner.

A recombinant protein may be conveniently prepared by a person skilled in the art using standard protocols as for example described in Sambrook et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

By “purify”, “purified” and “purification”, particularly in the context of recombinant protein purification, is meant enrichment of a recombinant protein so that the relative abundance and/or specific activity of said recombinant protein is increased compared to that before enrichment.

By “chromatography” such as in the context of chromatographic steps of the invention, is meant any technique used for the separation of biomolecules (eg protein and/or nucleic acids) from complex mixtures that employs two phases: a stationary bed phase and a mobile phase that moves through the stationary bed. Molecules may be separated on the basis of a particular physicochemical property such as charge, size, affinity and hydrophobicity, or a combination thereof.

The galectin-2 protein(s) of the present invention are particularly suited to ligand-based affinity chromatography using a β-galactoside such as lactose, conjugated to solid support, although without limitation thereto.

Chromatography may be performed by a person skilled in the art using standard protocols as for example described in CURRENT PROTOCOLS IN PROTEIN SCIENCE Eds. Coligan et al., (John Wiley & Sons, Inc. 1995-1999) which is incorporated by reference herein, in particular Chapters 8 and 9.

A preferred method of producing a recombinant galectin-2 mutant and/or derivative protein of the present invention includes the steps of:

(a) expressing a galectin-2 protein as a recombinant protein in a prokaryotic cell; and

(b) purifying the recombinant galectin-2 protein using an α-lactose-agarose column.

It is also contemplated that the isolated modified proteins, mutant, derivatives, variants and the like of the present invention may be produced by solid or liquid phase chemical synthesis as are well known in the art. By way of example, the skilled person is referred to 18 of CURRENT PROTOCOLS IN PROTEIN SCIENCE Eds. Coligan et al. John Wiley & Sons NY USA (1995-2001) for techniques applicable to chemical synthesis.

The invention also contemplates an antibody raised against an isolated galectin-2 protein or fragment as hereinbefore described.

In a preferred embodiment, the antibody binds to and/or has been raised against an isolated galectin-2 protein of the present invention whilst not binding, or binding with relatively lower affinity, to a wild-type galectin-2 protein.

Antibodies may be monoclonal or polyclonal, obtained for example by immunizing a suitable production animal (e.g. a mouse, rat, rabbit, sheep, chicken or goat). Serum or spleen cells may be then isolated from the immunized animal according to whether polyclonal or monoclonal antibodies are required.

Monoclonal antibodies may be produced by standard methods such as described in CURRENT PROTOCOLS IN IMMUNOLOGY (Eds. Coligan et al. John Wiley & Sons. 1995-2000) and Harlow, E. & Lane, D. Antibodies: A Laboratory Manual (Cold Spring Harbour, Cold Spring Harbour Laboratory, 1988). Such methods generally involve obtaining antibody-producing cells, such as spleen cells, from an animal immunized as described above, and fusing spleen cells with an immortalized fusion partner cell.

Recombinant antibodies are also contemplated. Selection of appropriate recombinant antibodies can be achieved by any of a number of methods including phage display, microarray or ribosome display, such as discussed in Hoogenboom, 2005, Nature Biotechnol. 23 1105, by way of example.

Also contemplated are antibody fragments such as Fab, F(ab)2, Fv, scFV and Fc fragments as well understood in the art.

As is also well understood in the art, in order to assist detection of antibody-antigen complexes, antibodies may be conjugated with labels including but not limited to a chromogen, a catalyst, an enzyme, a fluorophore, a chemiluminescent molecule, biotin and/or a radioisotope.

In other broad aspects, the invention provides a method of producing an isolated modified galectin-2 protein for use in a pharmaceutical composition with the method including the steps of mutating a galectin-2 protein at cysteine 57 or an analogous residue and/or modifying an isolated galectin-2 protein at cysteine 75 or an analogous residue, with respect to a wild-type galectin-2 protein, using methodology that is well known in the art and herein described.

It will be appreciated by a skilled addressee that in certain preferred embodiments that contemplate an isolated galectin-2 protein comprising a mutation at cysteine 57 and a modification at cysteine 75 on the same polypeptide, an isolated galectin-2 protein mutated at cysteine 57 may subsequently undergo a modification at cysteine 75 to thereby produce an isolated modified galectin-2 for use as a pharmaceutical composition. A non-limiting example of such a method is provided in the Examples section.

In other preferred embodiments, an isolated galectin-2 may be first modified at cysteine 75 and then undergo mutagenesis at cysteine 57 to thereby produce an isolated modified galectin-2 for use as a pharmaceutical composition.

### Pharmaceutical Compositions and Methods of Therapy

The invention contemplates methods of modulating an immune response in treatment of diseases, disorders or conditions in which galectin-2 has a therapeutic or potential therapeutic role. More particularly, the invention contemplates using the galectin-2 protein mutants and/or derivatives, as described herein. It will be appreciated that the methods of treatment of the present invention can be either prophylactic or therapeutic.

Although not wishing to be bound by any particular theory, galectin-2 is able to induce apoptosis of activated, but not resting T cells, down-regulates pro-inflammatory cytokine secretion, and blocks the adhesion of activated T cells to the extracellular matrix (8). Although it has been reported that galectin-1 could also induce T cell apoptosis, galectin-2 functions in a different way. In regard to galectin-1, its selection of distinct glycoprotein ligands, including CD3, CD7, CD43 and CD45, and its cross-linking ability, appear to be crucial for its induction of T cell apoptosis (9-12). In contrast to galectin-1, galectin-2 triggers T cell apoptosis via binding to T cells in a β-galactoside-specific manner, lacking reactivity with CD3 and CD7 (8). The potential therapeutic effects of galectin-2 in vivo have been further investigated in experimental colitis with a murine model (13). The study showed that galectin-2 was constitutively expressed mainly in the epithelial compartment of the mouse intestine and bind to lamina propria mononuclear cells (LPMC). Treatment with galectin-2 induced mucosal T cell apoptosis and thus ameliorated acute and chronic colitis. The results demonstrated that in LPMC, galectin-2 treatment reduced IL-12 p70 and IL-6 secretion and increase IL-10 secretion. IL-12 drives mucosal inflammation in many models of experimental colitis and Crohn's disease (CD). IL-6 is a potent pro-inflammatory cytokine which is probably centrally involved in the pathogenesis of IBD (14,15). IL-6 is also identified to be involved in the generation of a new T-cell subpopulation, Th17, which plays a predominant role in the pathogenesis of experimental autoimmune encephalomyelitis (16), auto immune arthritis (17), colitis due to IL-10 deficiency (18), and chronic colitis (19). All results indicate that galectin-2 might be an effective therapeutic agent for both acute and chronic IBD.

Apart from IBD, galectin-2 is also a binding and regulatory partner of lymphotoxin-α (LTA, also known as TNF-β), a pro-inflammatory cytokine. LTA has multiple functions in regulating the immune system and may contribute to inflammatory process leading to myocardial infarction (MI) (20), coronary heart disease (CHD) (21), and coronary artery disease (CAD) (22). The regulatory mechanism of LTA secretion could be that galectin induces T cell apoptosis and thus affects cytokine secretion (8). Ozaki et al. (20) found the genetic substitution affects the transcriptional level of galectin-2 in vitro, potentially resulting in altered secretion of LTA, thus affecting the degree of inflammation. The results indicated LTA and galectin-2 may have roles in the pathogenesis of MI (20). Also in a CHD study (21), it was concluded that an association between LTA and galectin-2 gene polymorphisms and markers of inflammation and cell adhesion molecules was evident.

In light of the foregoing, it will be appreciated that the invention contemplates methods of modulating an immune response and in preferred embodiments, an inflammatory immune response, by administering pharmaceutical compositions comprising isolated galectin-2 mutants and/or galectin-2 derivatives of the present invention. In preferred embodiments, the isolated galectin-2 mutant is C57M galectin-2 and the galectin-2 derivative is PEGylated Cys75.

In one preferred embodiment, the immune response is mediated by one or more cytokines or other soluble immunomodulators such as, but not limited thereto, CpG DNA, lipopolysaccharide (LPS) and leukotrienes. It will be appreciated that since galectin-2 is a potential mediator of pro-inflammatory responses by binding LTA, in particularly preferred embodiments, the one or more cytokines is LTA. Diseases, disorders or conditions that involve galectin-2 regulated secretion of LTA can be selected from the group consisting of myocardial infarction, coronary heart disease and coronary artery disease.

In other preferred embodiments, the invention contemplates modulating an immune response which is mediated by one or more cells of the immune system. In preferred embodiments, the one or more cells of the immune systems are activated T cells.

Therefore it will be appreciated that in certain preferred embodiments, the invention contemplates treatment of diseases, disorders or conditions responsive to inhibition or suppression of T cell activation using the galectin-2 protein mutants and/or derivatives, as described herein.

By “responsive to inhibition or suppression of T cell activation” is meant a disease, disorder or condition characterised by a shift in T cell homeostasis towards at least T cell activation, and in some circumstances, T cell hyperactivation. In light of the foregoing, it will be appreciated that galectin-2 is suited for the therapeutic use in treatment of diseases with impaired T cell apoptosis. Galectin-2 also has an immunomodulatory capacity and potential to maintain inflammatory and autoimmune responses in check. For example, galectin-2 may be a particularly useful therapeutic agent where restoration of T cell homeostasis is desirable. Non-limiting examples include allergic reaction, graft or transplant rejection by a host such as is common in graft-versus-host-disease and forms of cancer.

The invention contemplates treatment of both systemic and organ-specific autoimmune diseases. Systemic autoimmune diseases include rheumatoid arthritis and lupus, but without limitation thereto. The invention contemplates other organ-specific autoimmune diseases such as, but without limitation thereto, autoimmune hepatitis and endocrine-specific autoimmune diseases.

The invention is also suited to gastrointestinal-related inflammatory diseases such as inflammatory bowel disease (IBD). In animal model studies based on mice, galectin-2 has been linked with IBD (Paclik et al, J Mol Med, 2007 Dec. 7). IBD can be generally classed as an autoimmune diseases and is inclusive of Crohn's Disease (CD), ulcerative colitis (UC) and indeterminate colitis (IC). IC is a medical term referred to when discrimination between CD and UC can not be made with certainty.

In preferred embodiments, the diseases, disorders or conditions responsive to inhibition or suppression of T cell activation are selected from the group consisting of IBD, an allergic reaction and graft-versus-host-disease.

The pharmaceutical composition of the present invention may conveniently be provided or formulated in a pharmaceutical composition comprising the isolated proteins and/or isolated nucleic acids of galectin-2 and a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines. In the case of gene therapy, which contemplates the use of electroporation or liposomal transfection into tissues, the drug may be transfected into cells together with the DNA.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgment of the practitioner.

So that the invention may be readily understood and put into practical effect, reference is made to the following non-limiting Examples.

It will also be appreciated that treatment methods and pharmaceutical compositions may be applicable to prophylactic or therapeutic treatment of mammals, inclusive of humans and non-human mammals such as livestock (e.g. horses, cattle and sheep), companion animals (e.g. dogs and cats), laboratory animals (e.g. mice rats and guinea pigs) and performance animals (e.g racehorses, greyhounds and camels), although without limitation thereto.

### EXAMPLES

### Example 1

### A Single Cysteine Mutation of Human Galectin-2 Confers Enhanced Aggregation Stability and Enables Site-Directed MonoPEGylation

### Materials and Methods

Site-Directed Mutagenesis and Expression of hGal2.

hGal2 (Swiss-Prot ID: P05162) was encoded in plasmid pQE60 (Qiagen, Hilden, Germany). The following oligonucleotides were used for site-directed mutagenesis of Cys57 (FIG. 1):

Cys57Met-upstream primer

| | |
|---|---|
| 5′ CCACCATTGTCATGAACTCATTGGAC 3′ | (SEQ ID NO: 6) |

and
downstream primer

| | |
|---|---|
| 5′ GTCCAATGAGTTCATGACAATGGTGG 3′; | (SEQ ID NO: 7) |

Cys57Ala-upstream primer

| | |
|---|---|
| 5′ CCACCATTGTCGCGAACTCATTGGAC 3′ | (SEQ ID NO: 8) |

and
downstream primer

| | |
|---|---|
| 5′ GTCCAATGAGTTCGCGACAATGGTGG 3′; | (SEQ ID NO: 9) |

Cys57Ser-upstream primer

| | |
|---|---|
| 5′ CCACCATTGTCTCCAACTCGTTGGAC 3′ | (SEQ ID NO: 10) |

and
downstream primer

| | |
|---|---|
| 5′ GTCCAACGAGTTGGAGACAATGGTGG 3′. | (SEQ ID NO: 11) |

Plasmid DNA was sequenced to confirm the site-directed mutagenesis of hGal2 and then transformed into E. coli BL21 (DE3). For expression, an aliquot of the glycerol stock was streaked on an LB agar plate containing 100 μg/mL of ampicillin and incubated overnight at 37° C. A single colony was selected and used to inoculate a 5 mL LB (Luria broth) medium containing 100 μg/mL of ampicillin. This 5 mL culture was agitated overnight using a horizontal shaker (Rowe Scientific Pty Ltd, Perth, Australia) set at 180 rpm and 37° C. 100 μL of overnight culture was subsequently added into 500 mL LB medium supplemented with fresh 100 μg/mL ampicillin. This 500 mL culture was induced with 1 mM IPTG when the OD₆₀₀ reached 1.0±0.1. Expression was terminated after 4 h of induction when the OD₆₀₀ was 2.0-2.2. Cell pellets were harvested using a Sorvall® Super T21 centrifuge (Thermo Scientific, MA, USA) at 2000×g, 4° C., 30 min, washed in PBS buffer A (2 mM KCl, 137 mM NaCl, 10 mM Na₂HPO_{4,} pH 7.0), centrifuged again (as above) and stored at −20° C. until required.

For solubility analysis, B-PER® bacterial protein extraction reagent (Pierce, Ill., USA) was used according to the manufacturer instructions. Briefly, 1.5 mL bacterial culture (OD₆₀₀=2) was centrifuged at 5000×g for 10 min. Cells were resuspended in 300 μL of B-PER® reagent and centrifuged again at 15000×g for 5 min. Supernatant (soluble fraction) was transferred to a new tube and the pellet (insoluble fraction) was resuspended in 300 μL of B-PER® reagent for further analysis.

### Agilent 2100 Bioanalyzer Analysis.

hGal2 expression and solubility were estimated using a chip-based separation technique, performed on an Agilent 2100 Bioanalyzer™ (Agilent, Forest Hill, Australia) in combination with the Protein 80 Plus LabChip® kit. Briefly, 4 μL of sample was mixed with 2 μL of sample buffer which included an upper marker that can be used for semi-quantitative analysis. Samples and the standard protein ladder provided with the LabChip® kit were heated at 95° C. for 5 min and diluted with 84 μL Milli-Q water. After brief centrifugation, samples and standard protein ladder were loaded onto a chip filled with gel-dye mixture. Separated proteins were detected by laser-induced fluorescence.

Affinity Purification of hGal2.

Cell pellet from 2 L of culture was resuspended in 50 mL PA buffer (20 mM sodium phosphate, 1 mM EDTA, 100 mM NaCl, 5 mM DTT, pH 8.0) and then passed once through a homogenizer (Niro Soavi S. p. A., Parma, Italy) at 1000 bar. Cell debris was removed by centrifugation (10000×g, 4° C., 30 min). hGal2 purification from the supernatant used affinity chromatography with lactosylated agarose (25). 15 mL supernatant was loaded on an Omnifit column (inner diameter I.D. 10 mm, Cambridge, UK) packed with 7 cm α-lactose-agarose (Sigma-Aldrich, Sydney, Australia) and washed with 5 mL PA buffer. Purified hGal2 was eluted with 33% PB buffer (20 mM sodium phosphate, 1 mM EDTA, 100 mM NaCl, 5 mM DTT, 300 mM Lactose, pH 8.0). All process chromatography experiments were performed on an ÄKTAexplore workstation (GE Healthcare, Sydney, Australia) at room temperature. Collected fractions were analyzed on the Bioanalyzer® 2100 system, as described above.

N-Terminal Sequencing of hGal2 C57M

N-terminal sequencing of hGal2 C57M was performed by the Australian Proteome Analysis Facility (Sydney, Australia). 100 μL of the sample at a concentration of 1 mg/mL was desalted and placed onto a polyvinylidene fluoride (PVDF) membrane using a ProSorb cartridge with 2×200 μL washes with 0.1% trifluoroacetic acid (TFA). The sample was then subjected to 7 residue cycles of Edman N-terminal sequencing, using an Applied Biosystems 494 Precise Protein Sequencing System. The performance of the sequencer is assessed routinely with 10 pmol β-Lactoglobulin standard.

### Liquid Chromatography-Mass Spectrometry (LC-MS).

API QStar Pulsar mass spectrometer system (quadrupole-time-of-flight) with an electrospray ionization source (MDS Sciex, Ontario, Canada) was used to analyze protein samples. 100 μL of 0.8-1 mg/mL protein solution was desalted on a C18 column using a linear acetonitrile-water gradient with 0.1% (v/v) ormic acid (starting at 0% (v/v) acetonitrile to a final concentration of 72% in 42 min). The spectra were analyzed using Analyst software (version 1.1).

PEGylation of hGal2 C57M and Post-Reaction Purification.

Maleimide-PEG 5 kDa (average molecular weight Mn 5,522, polydispersity (Mw/Mn) 1.03) from NOF Corporation (Tokyo, Japan) was selected as the PEG-thiol reagent. Purified hGal2 C57M was firstly desalted into PEGylation buffer (20 mM sodium phosphate, 100 mM NaCl, 100 mM Lactose, pH 6.8) using an Omnifit column (inner diameter I.D. 15 mm, Cambridge, UK) packed with 12 cm Sephadex G25 gel matrix (GE Healthcare, Sydney, Australia). A known amount of PEG 5-kDa was then added to the protein solution (typical hGal2 concentration of 1-2 mg/mL) to give a final PEG:hGal2 molar ratio of 3:1 (equal to a mass ratio of 1.1:1). The solution was mixed, and left to react overnight at 4° C. The reaction product was desalted into IA buffer (20 mM Tris, 100 mM Lactose, pH 7.5), before loading onto a 5-mL HiTrap QFF column (GE Healthcare, Sydney, Australia). Two-step elution was conducted as follows: in the first elution step, purified-PEGylated hGal2 C57M was eluted with 8% IB Buffer (20 mM Tris, 100 mM Lactose, 1M NaCl, pH 7.5). Remaining contaminants were eluted with a linear gradient of 8-100% IB buffer in 10 min. The purified-PEGylated hGal2 C57M was finally desalted into the desired buffer condition (PBS with or without 100 mM lactose) using the Sephadex G25 gel-filtration column.

MALDI-TOP MS Analysis of PEGylated hGal2 C57M

PEGylated hGal2 C57M was analyzed by matrix assisted laser adsorption ionization (MALDI) time of flight (TOF) mass spectrometry, using a Bruker Daltonics Microflex MALDI-TOF Mass Spectrometer at the Australian Proteome Analysis Facility Ltd. Sample was prepared as follows: 0.3 μL of 0.5 mg/mL PEGylated hGal2 C57M was spotted onto a sample plate with 1 μL of matrix (α-cyano-4-hydroxycinnamic acid, 6 mg/mL in 70% v/v MeCN, 0.06% v/v TFA, 1 mM ammonium citrate) and allowed to air dry. The sample was then desalted three times with 0.1% TFA, and dried under vacuum prior to analysis.

### CD Spectra.

Far-UV (200-250 nm) CD spectra of hGal2 WT, hGal2 C57M, and PEGylated hGal2 C57M were obtained using a Jasco-810 spectropolarimeter (Jasco Corp., Tokyo, Japan). Cell path lengths were 1 mm for far-UV measurements. Prior to the measurement, protein samples were desalted into 1 mM HEPES buffer at pH 7.2. Protein (300 μL at 0.5-0.8 mg/mL) was analyzed at room temperature, 23±2° C. Spectra were corrected by subtracting the buffer baseline, and were averaged over 10 scans for all measurements, using 1-nm intervals, a response time of 2 sec, and 1-nm bandwidth.

### Surface Plasmon Resonance (SPR).

A SPR system with a Kretschmann configuration (26) was from Resonant Probes GmbH (Goslar, Germany). The binding ability of hGal2 with asiolofetuin (ASF) before and after PEGylation was examined with a home-made ASF-SPR chip. This ASF-SPR chip has a multilayer (thiol-SAM)/Biotin/Streptavidin (SA)/ASF architecture (FIG. 2). The Biotin-SA layer serves as a matrix for the ASF ligand, providing an interfacial architecture having molecularly-controlled order and orientation, thus minimizing non-specific adsorption (27).

The ASF-SPR chip was prepared starting with a 50 nm gold-coated SF10 glass substrate. The first SAM layer was self-assembled using a mixed ethanolic solution of 5×10⁻⁵ M cysteamine (NH₂ terminated thiol) (Sigma-Aldrich, Sydney, Australia) and 4.5×10⁻⁴ M mercaptoethanol (OH terminated thiol, as spacers) (Sigma-Aldrich, Sydney, Australia) for 15 h at room temperature. The second layer of biotin was introduced by coupling biotin-hexanoic acid (Sigma-Aldrich, Sydney, Australia) with cysteamine by activating 1 mg/ml biotin-hexanoic acid in PBS (prepared from a 6 mg/mL stock in dimethylformamide (DMF, Sigma-Aldrich, Sydney, Australia) with 4 mg/mL of 1-(3-dimethylaminopropy)-3-ethylcarbodiimide hydrochloride (EDC, Sigma-Aldrich, Sydney, Australia) and 0.7 mg/mL of N-hydroxysuccinimide (NHS, Sigma-Aldrich, Sydney, Australia). The third layer (of SA) was constructed by loading 45 μg/mL of SA (Invitrogen, Mount Waverley, Australia) into the flow cell, which specifically binds to biotin. This step took about 10 min to reach equilibrium. The SA layer was then activated by 4 mg/mL EDC and 0.7 mg/mL NHS for 5 min, enabling its carboxylic acid residue to react with the primary amino group of ASF (Sigma-Aldrich, Sydney, Australia). 1.0 mg/mL ASF was used in this step to prepare the fourth layer. Non-specific binding of proteins to the ASF-SPR chip was tested by adsorption of 4.4 mg/mL of bovine serum albumin (BSA, Sigma-Aldrich, Sydney, Australia).

Prior to hGal2 binding tests, the ASF-SPR chip was equilibrated with PBS. The binding of hGal2 onto the chip was continuously recorded by monitoring the reflectivity at a fixed angle (ca. 60°, giving an initial reflectivity about 30%). Protein solutions at increasing concentration (0.3-4 mg/mL) were sequentially loaded into the flow cell. After the highest concentration sample was loaded and equilibrium was reached, the flow cell was washed extensively with PBS.

### Stability Investigation.

As described above, the PEGylated hGal2 was purified from the reaction solution by ion-exchange chromatography. In order to standardize sample buffer conditions for stability testing, hGal2 WT and C57M samples after affinity purification were also subject to one additional IEC purification step, following the same procedure as for PEGylated hGal2. All protein solutions from the IEC column were then desalted into PBS using a desalting column (15 mm×120 mm, packed with Sephadex G25). The protein solutions at a concentration of 1 mg/mL were stored at 4° C. over 3 weeks. From time to time, samples were taken to determine the extent of aggregation using gel-filtration chromatography. Typically, 80 μL of sample was loaded onto a Superdex™ 200 GL column (10×300 mm, GE Healthcare, Sydney, Australia), followed by elution with PBS at a flow rate of 0.5 mL/min.

### Results

### Mutagenesis, Expression, and Purification of Human Galectin2.

In this work, three mutant hGal2 designs were engineered by substituting the Cys57 residue with three different residues (C57M, C57A, and C57S). DNA sequencing confirmed successful mutation. Our wild type DNA sequence was compared with sequence Swiss-Prot ID P05162 which was expressed in pET-11b/E. coli BL21 cells (1). The results showed our hGal2 WT was identical to P05162 except for the first 2 amino acid residues (N-terminal sequence AGELEVK (SEQ ID NO:12) versus MTGELEVK (SEQ ID NO:13) for P05162). N-terminal sequencing of the final C57M protein reported here confirmed this variation.

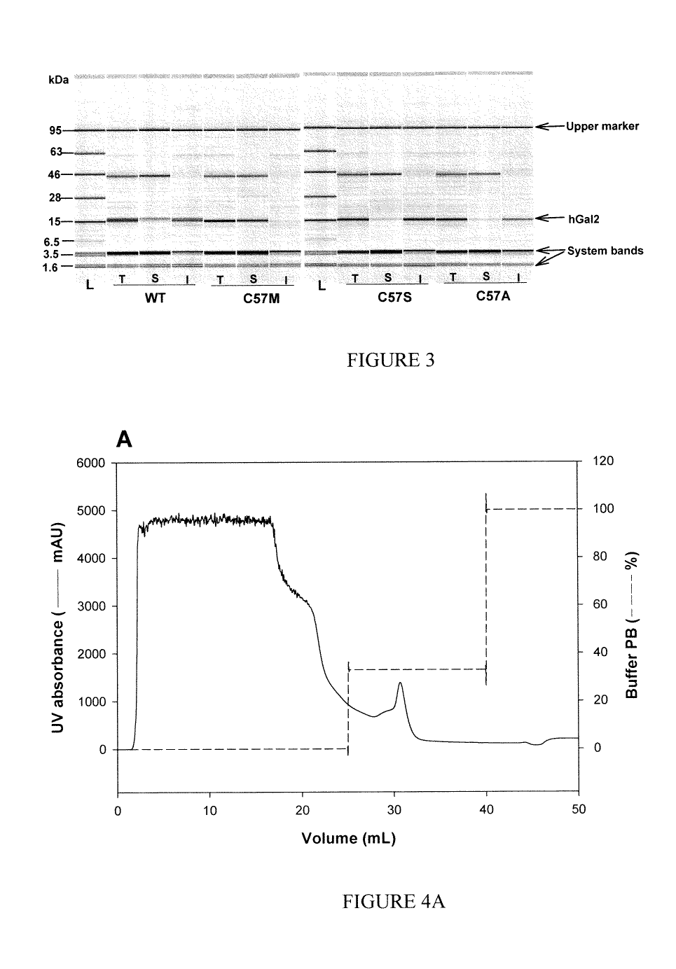

Wild type hGal2 and mutants expressed in BL21 (DE3) were compared for total expression, soluble fraction and insoluble fraction. As shown in FIG. 3, C57S and C57A were predominantly insoluble, suggesting these two mutants tend to form aggregates. In contrast to these two mutants, the majority of C57M was soluble. Furthermore, the soluble expression level of C57M was higher than that of wild type, suggesting that the C57M mutant will have better processing characteristics than the wild type hGal2.

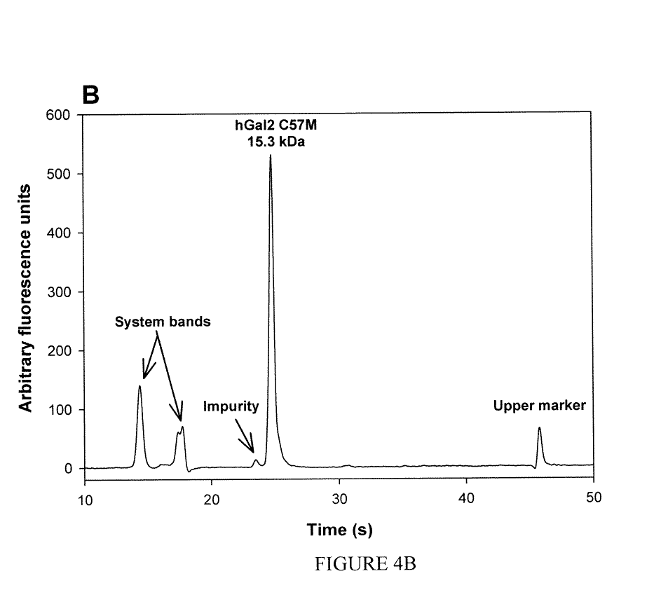

Both hGal2 WT and C57M purification were based on affinity binding of gal-2 with lactose (25). A typical purification result is shown in FIG. 4, using C57M as an example. The binding of hGal2 onto lactose-agarose is not very strong due to relatively weak interaction between hGal2 and lactose (dissociation constant k_{d}=68 μM (28)); this weak interaction caused a loss of bound protein during the washing step, and chromatogram tailing was observed. Purified hGal2 was eluted with 100 mM lactose in the first elution step (FIG. 4A) and was analysed using a Bioanalyzer® 2100 (FIG. 4B), which shows that the recovered C57M had a purity of approximately 97% with a measured molecular weight 15.3 kDa (theoretical MW 14.5 kDa according to sequence), similar to that of wild type (data not shown).

### Characterization of Human Galectin2.

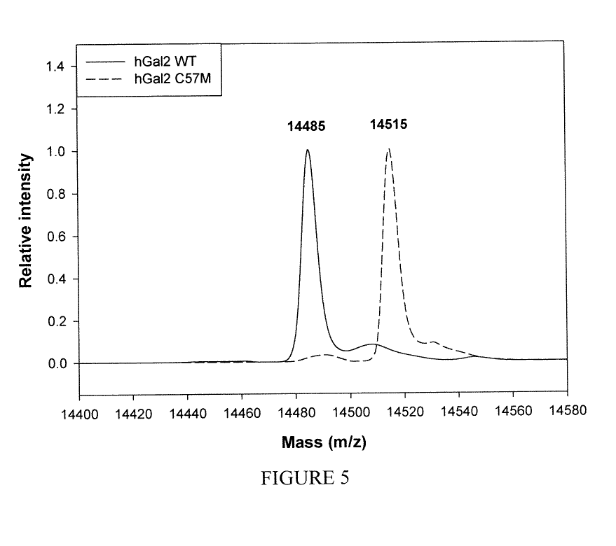

LC-MS analysis was performed to characterize the hGal2 WT and C57M. As shown in FIG. 5, the mass spectra presented peaks at masses 14485 Da and 14515 Da for hGal2 WT and C57M, respectively. The theoretical mass of our recombinant hGal2 WT and C57M, as calculated from their amino acid sequence using the ProtParam tool on the ExPASy server (http://ca.expasy.org/tools/protparam.html), were 14483 Da and 14511 Da, respectively. For both proteins, the difference of mass obtained from measurement and calculation was less than 4 Da, confirming the success of site-directed mutagenesis.

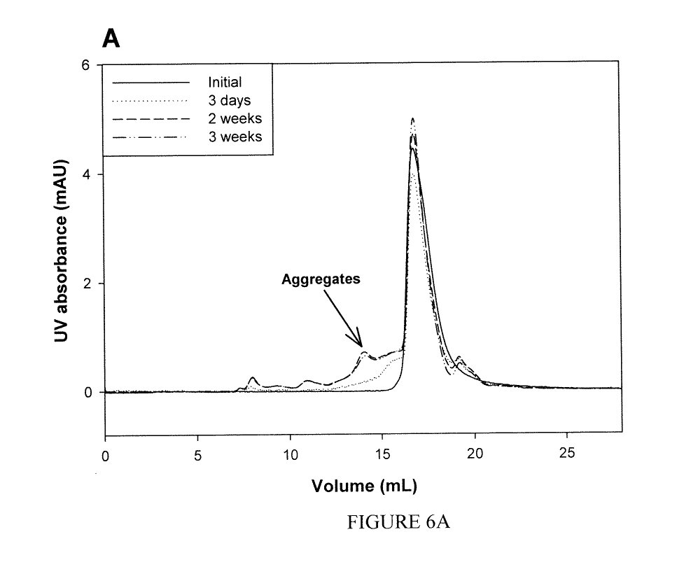

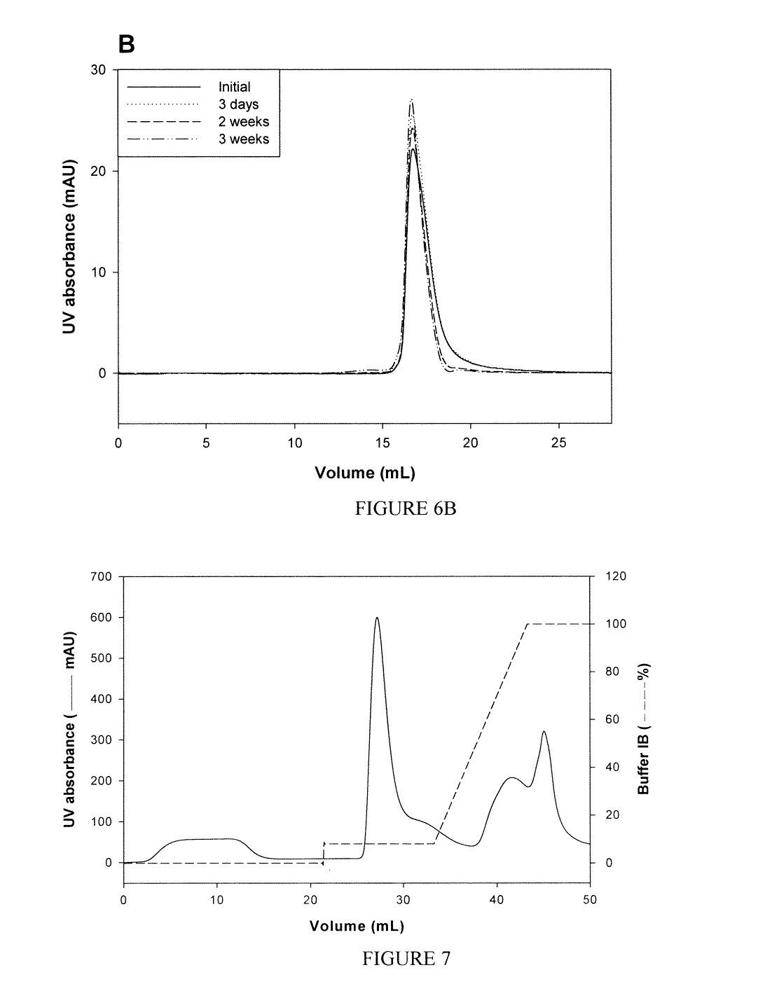

The storage stability of hGal2 WT and C57M were compared (3 weeks at 4° C.). hGal2 WT formed aggregates within 3 days and the extent of aggregation increased with storage time (FIG. 6A). After 3 weeks, a significant amount of hGal2 WT had been lost to aggregation. In contrast to WT, no detectable aggregation was observed for C57M after 3 weeks cold storage (FIG. 6B). The results suggest that hGal2 stability was significantly improved by the designed Cys57Met mutation.

PEGylation of hGal2 C57M

Maleimide-PEG 5-kDa was used as the PEG-thiol reagent. PEGylation was completed by incubating the mixture of PEG-thiol reagents and hGal2 C57M at 4° C. overnight. Control experiments proved that a PEG/hGal2 molar ratio of 3:1 is sufficient to ensure complete PEGylation of hGal2 (data not shown). Anion exchange chromatography was found to be a highly efficient method to separate PEGylated C57M from residual C57M and other contaminants. As shown in FIG. 7, PEGylated protein bound weakly to the column, compared to unmodified protein, and was eluted at 8% IB Buffer. The remaining contaminants were eluted at higher ionic strength.

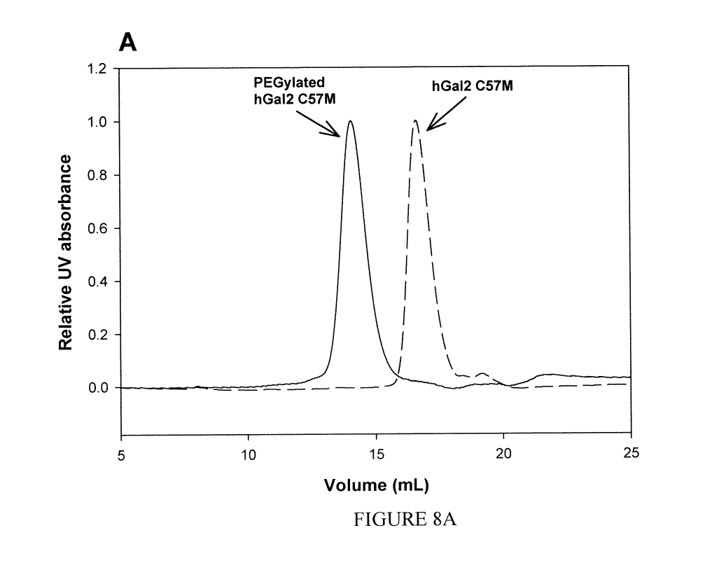

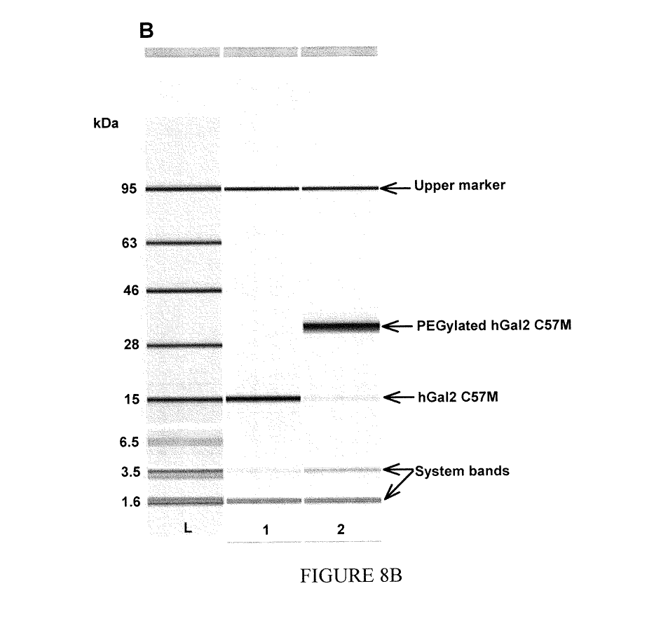

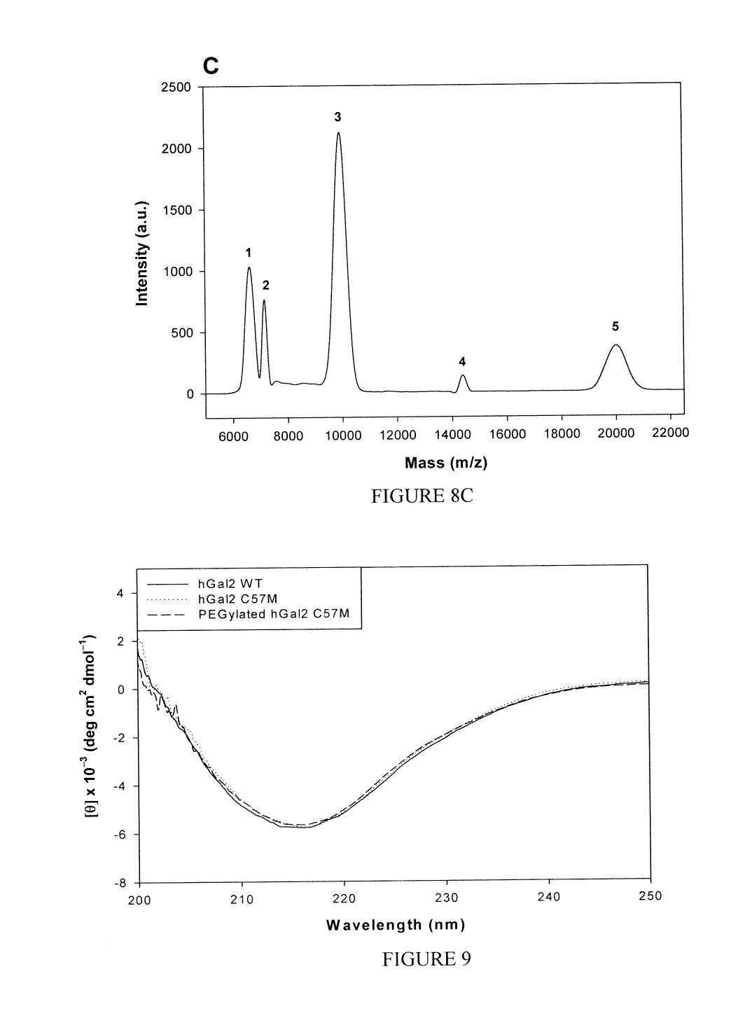

The size of the PEGylated C57M product was analysed using gel-filtration chromatography. As shown in FIG. 8A, the elution volume of proteins decreased from 16.7 mL to 14.1 mL after PEGylation, suggesting an increase in hGal2 size following PEGylation. The single narrow elution peak also suggests a high purity of the final product. It is known that galectin-2 exists as a dimer in the crystal phase (17). Our preliminary small angle X-ray (SAXS) and neutron (SANS) scattering data also suggest hGal2 is a homodimer in buffered solution (manuscript in preparation). In this study, although gel-filtration chromatography results suggest that PEG has been successfully conjugated to the dimer, it cannot be determined whether each monomer of the hGal2 dimer is PEGylated (PEG-Gal-Gal-PEG), or alternatively whether just one hGal2 monomer within the dimer is PEGylated (PEG-Gal-Gal), leaving another monomer unmodified. In order to confirm the final PEGylation product, PEGylated hGal2 C57M was tested using the Bioanalyzer® 2100. If the hGal2 dimer were heterogeneously PEGylated to yield PEG-Gal-Gal, analysis should reveal a large amount of unPEGylated hGal2 (around 15 kDa). As shown in FIG. 8B, the PEGylated product is characterised by one broad band around 34.5 kDa. The PEG used is 5500 Da, which presents as around 15 kDa due to its large exclusion volume. The band near 30 kDa is thus most likely PEGylated hGal2. More importantly, only a very narrow and light band around 15 kDa was observed, due to a small residual amount of unPEGylated hGal2. Thus the Bioanalyzer results suggest that both monomers of the hGal2 dimer were successfully PEGylated, yielding PEG-Gal-Gal-PEG.

To further characterize the final PEGylation products, PEGylated hGal2 C57M was analysed using MALDI-TOF MS (FIG. 8C). The MALDI-TOF MS spectra showed peaks corresponding to the single-charged ion of mono-PEGylated hGal2 C57M at mass 20045 (peak 5), the double-charged ion at mass 9953 (peak 3), and the triple-changed ion at mass 6634 (peak 1). The ions with mass 14398 (peak 4) and mass 7151 (peak 2) were due to the presence of a small amount (less than 9%) of the single- and double-charged ions of free hGal2 C57M. The difference in mass between hGal2 C57M and PEGylated hGal2 C57M was around 5600 Da, consistent with the molecular mass of PEG (5500 Da) used for PEGylation. More importantly, there were no other peaks representing multiPEGylated isomers, confirming that each hGal2 C57M molecule was indeed conjugated with a single PEG molecule.

Characterization of PEGylated hGal2 C57M.

In order to explore PEG's effect on the secondary structure of modified molecules, PEGylated hGal2 C57M was studied using far-UV circular dichroism (CD). As shown in FIG. 9, the CD scans of hGal2 WT, hGal2 C57M and PEGylated hGal2 C57M showed low intensity spectra with minima in the 215-217 nm range. The result is consistent with the CD spectra of galectin-1 (29), which also has a large extent of β-sheet structural profile as shown by X-ray crystallography (17,30). The near superposition of the far-UV spectra of these three samples suggests that mutation and then PEGylation did not cause significant perturbation of hGal2 secondary structure.

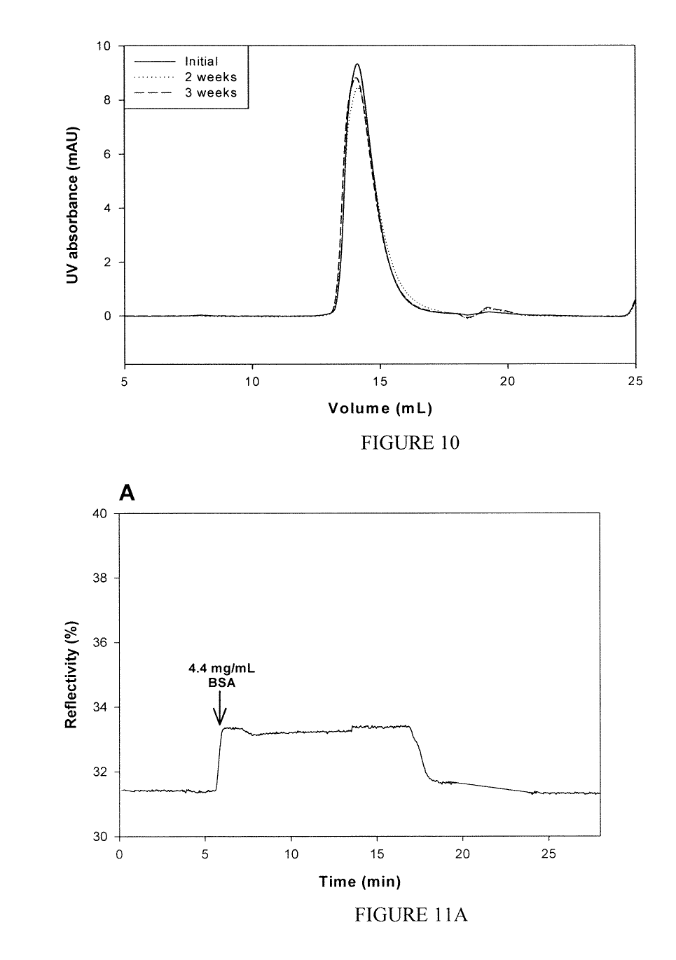

The storage stability of PEGylated hGal2 C57M was also assessed using size exclusion chromatography. The sample was stored in PBS at 4° C. over 3 weeks. As shown in FIG. 10. PEGylated hGal2 C57M was stable under this condition.

Binding Activity of hGal2.

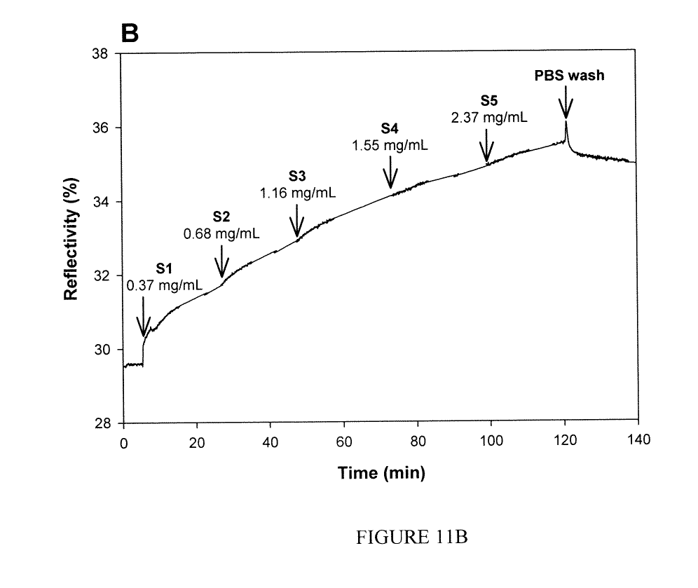

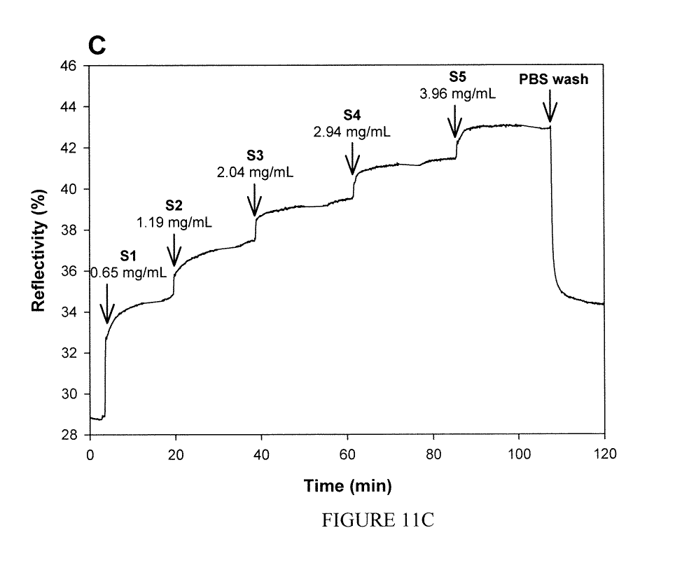

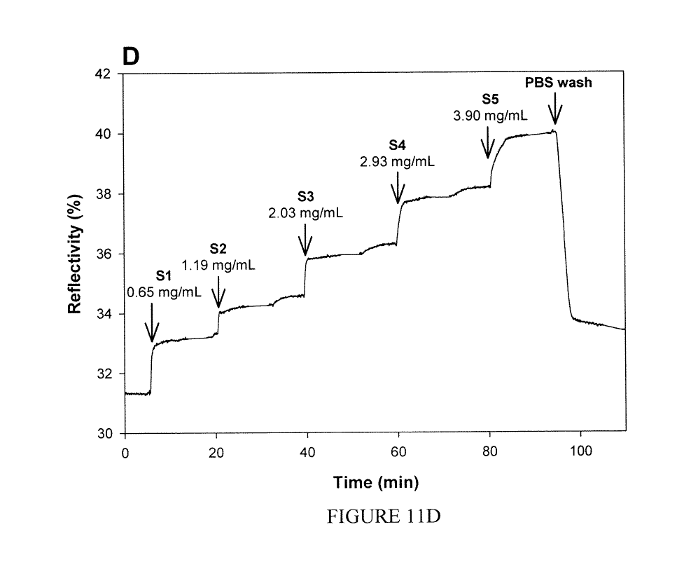

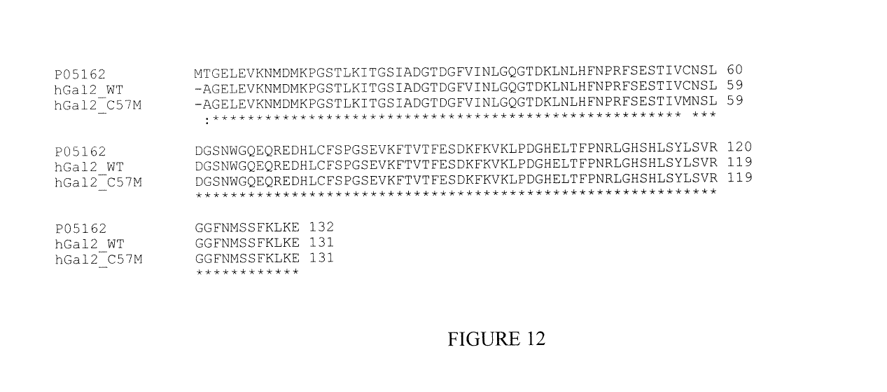

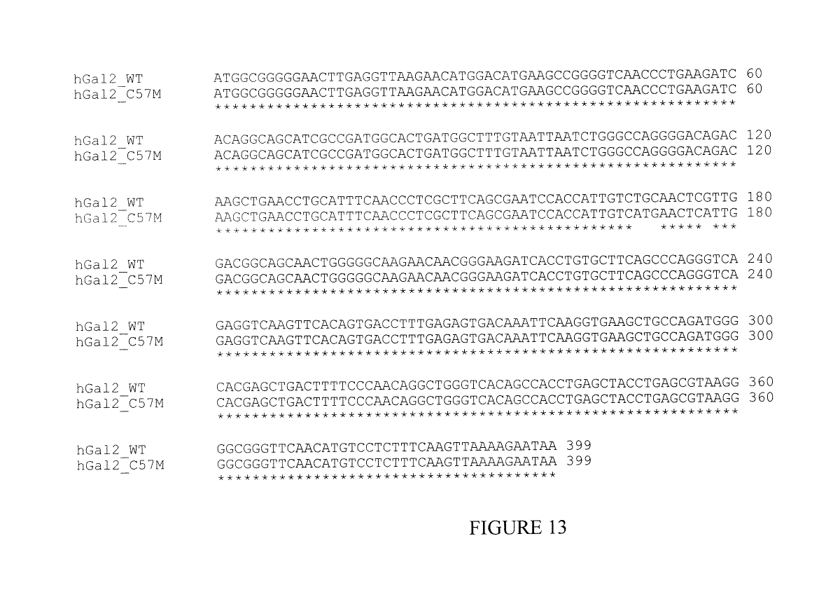

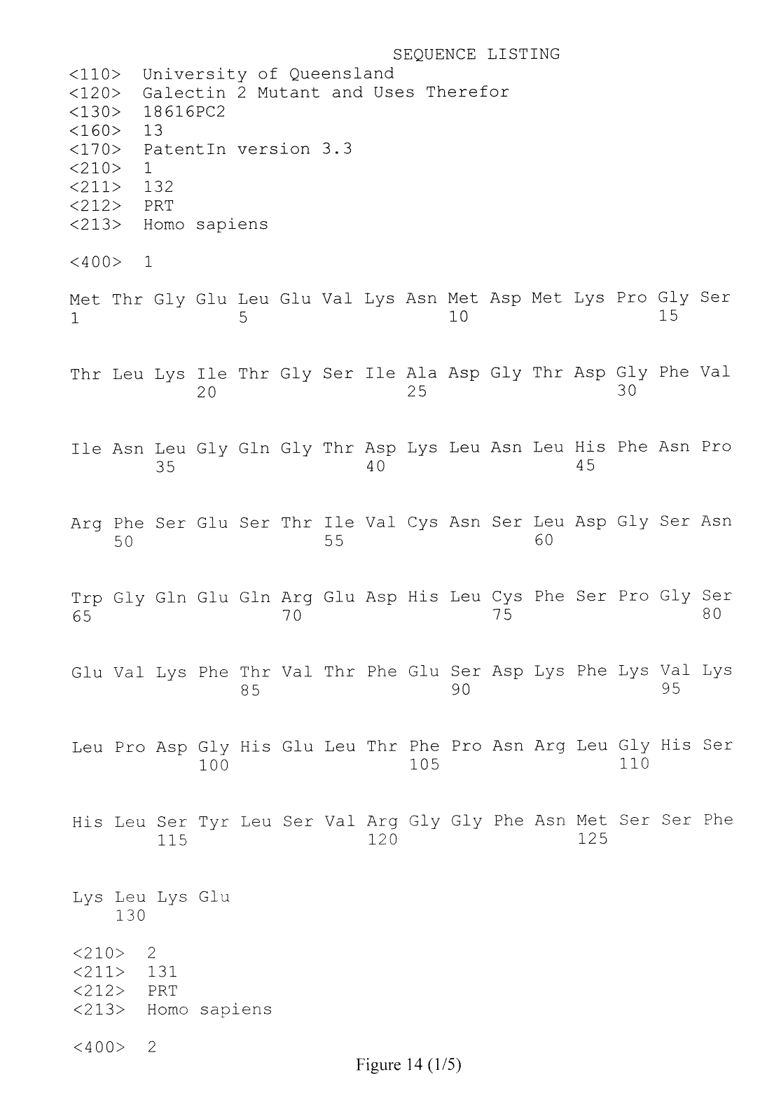

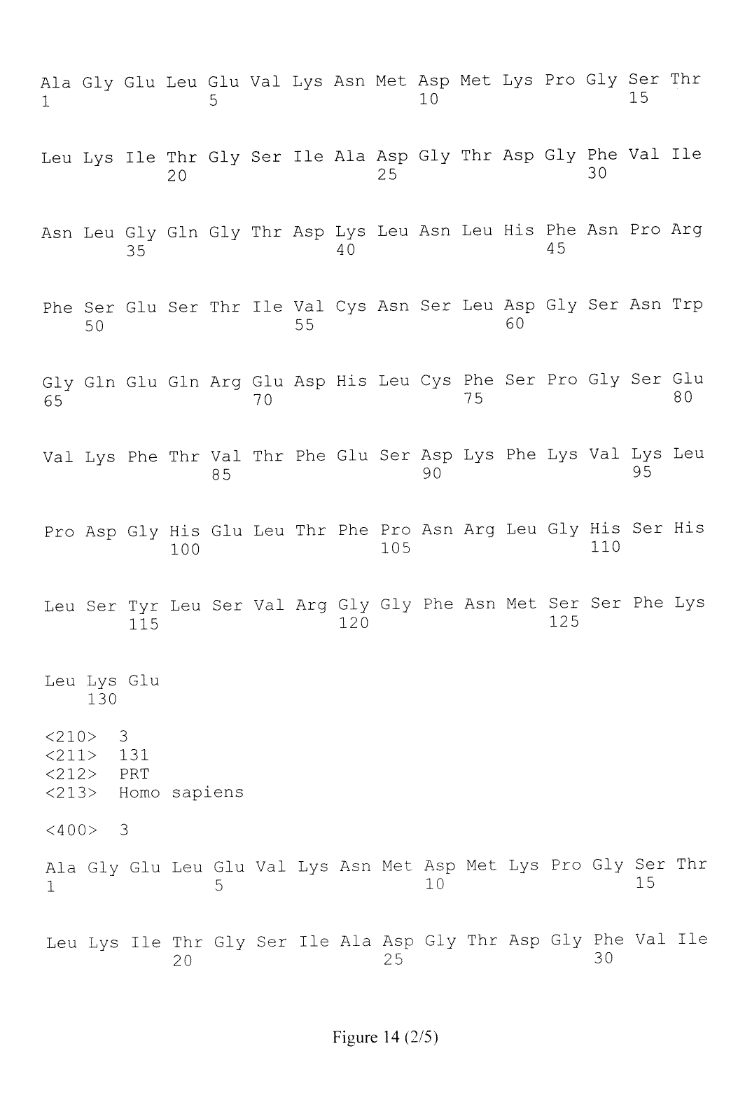

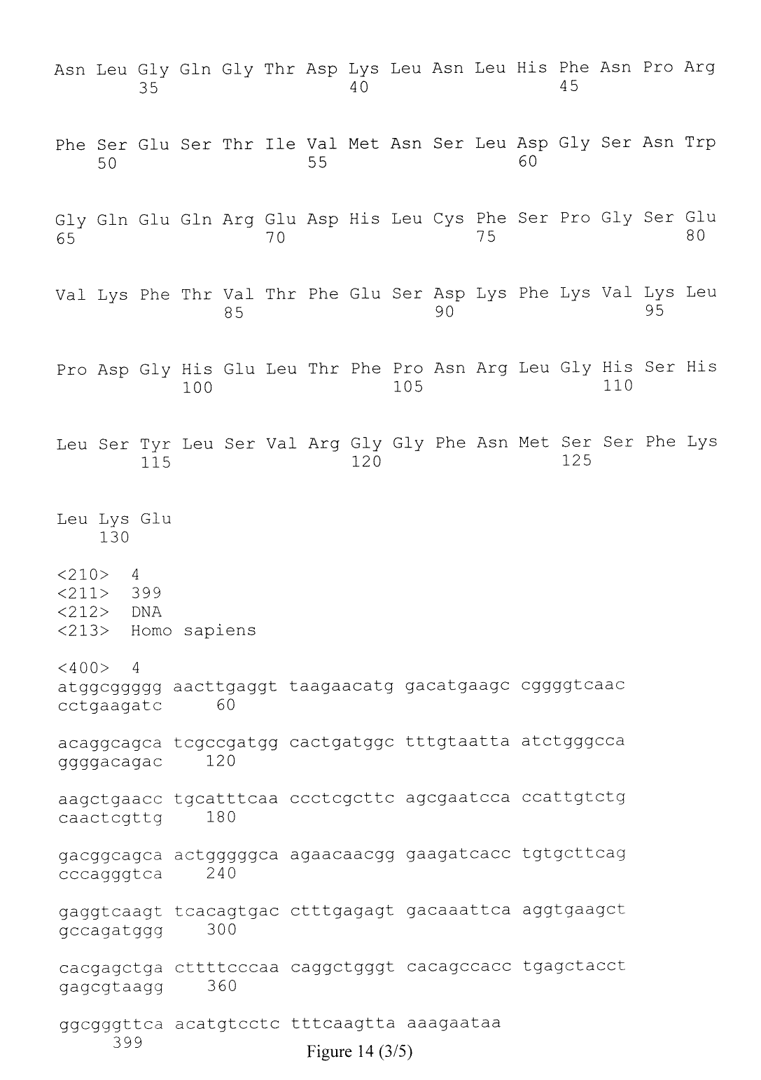

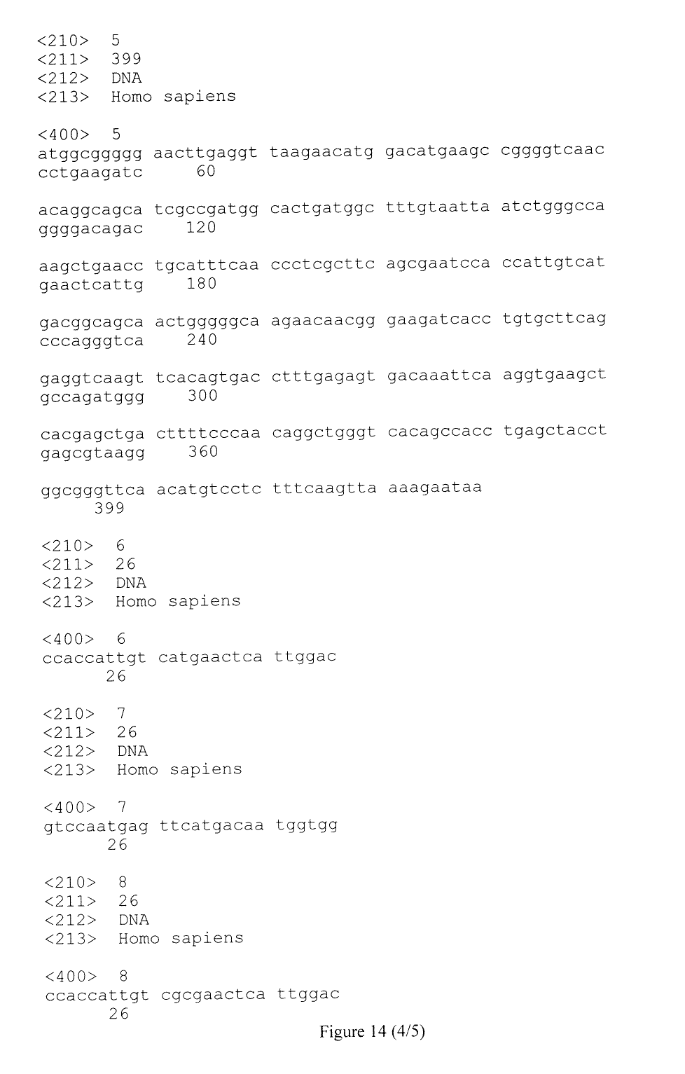

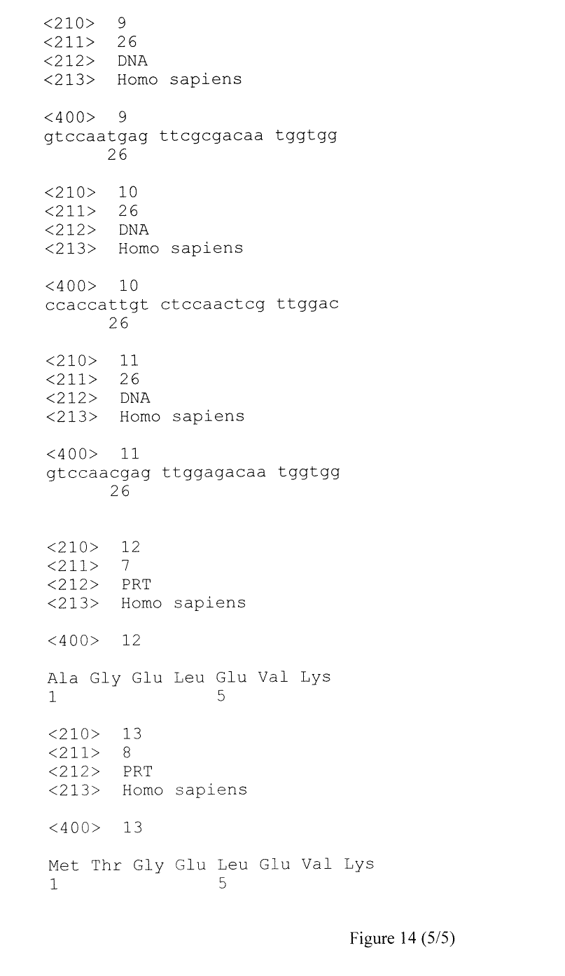

SPR with an asialofetuin (ASF)-immobilized chip was used to test hGal2 binding activity. ASF is a 48 kDa glycoprotein (31) possessing three triantennary N-link oligosaccharide chains with terminal N-acetyllactosamine (LacNAC) residues (32) and three O-linked disaccharide chains (33). It has been reported that kₐ values of ASF to galectins are 50-80-fold greater than for lactose (34). Non-specific binding to the designed ASF-SPR chip was first tested with 4.4 mg/mL of BSA. As shown in FIG. 11A, BSA did not cause significant irreversible adsorption on the surface, and could be completely and quickly washed away with PBS. In terms of hGal2 WT, hGal2 C57M and PEGylated hGal2 C57M, the reflectivity was significantly increased due to the binding of samples to the ASF-immobilized surface, even at low hGal2 concentration (0.3-0.65 mg/mL; FIG. 11B-D). Additionally, the adsorption of hGal-2, hGal2 C57M and PEGylated hGal2 C57M was partially irreversible. Even with intensive washing, some protein remained adsorbed on the surface, suggesting the binding of galectins to ASF was specific. Also SPR experiments demonstrated that mutation and PEGylation did not cause a loss of hGal2 ASF binding activity.

### Discussion

Galectins play key roles in the whole organism, such as regulation of immunity and inflammation (35,36), progression of cancer (37), and in tissue development (38,39).

Protein-glycan interactions control essential immunological process (1). Galectins, as a family of highly conserved glycan-binding proteins, play key roles as putative modulators of immune surveillance, apoptosis, cell adhesion and cytokine secretion (2). Although all members of the galectin family contain conserved carbohydrate-recognition domains (CRD), it has become increasingly clear that galectins can affect cellular activation and function in different ways. For example, galectin-1, which distributes in a wide variety of tissues, shows specific growth inhibitory properties toward different cell types, such as phytohemagglutinin (PHA)-activated human T cells (3,4), chicken activated lymphocytes (5) and other cell types (6). Even the same galectin may show opposite immunomodulatory effects on different cells types (7).

Early studies classified human galectin-2 as a proapoptotic effector for activated T cells. Galectin-2 can induce apoptosis of activated, but not resting T cells, down-regulates pro-inflammatory cytokine secretion, and blocks the adhesion of activated T cells to the extracellular matrix. Although it has been reported that galectin-1 could also induce T cell apoptosis, galectin-2 functions in a different way. Basically, for galectin-1, its selection of distinct glycoprotein ligands, including CD3, CD7, CD43 and CD45, and its cross-linking ability, appear to be crucial for its induction of T cell apoptosis. In contrast to galectin-1, galectin-2 triggers T cell apoptosis via binding to T cells in a β-galactoside-specific manner, lacking reactivity with CD3 and CD7. Recently the potential therapeutic effects of galectin-2 in vivo have been further investigated in experimental colitis with a murine model. The study showed that galectin-2 was constitutively expressed mainly in the epithelial compartment of the mouse intestine and bind to lamina propria mononuclear cells (LPMC). Treatment with galectin-2 induced mucosal T cell apoptosis and thus ameliorated acute and chronic colitis. The results demonstrated that in LPMC, Gal2 treatment reduced IL-12 p70 and IL-6 secretion and increase IL-10 secretion. IL-12 drives mucosal inflammation in many models of experimental colitis and Crohn's disease (CD). IL-6 is a potent pro-inflammatory cytokine which is probably centrally involved in the pathogenesis of IBD. IL-6 is also identified to be involved in the generation of a new T-cell subpopulation, Th17, which plays a predominant role in the pathogenesis of experimental autoimmune encephalomyelitis, auto immune arthritis, colitis due to IL-10 deficiency, and chronic colitis. Paclik et al. also investigated galectin-2 toxicity with mice model. Galectin-2 is well tolerated at a dose of 100 mg/kg BW Gal2 i.p. once daily, which is 50 times higher than the therapeutically most active concentration. All results indicate that galectin-2 might be a new therapeutic agent for both acute and chronic IBD.

Apart from IBD, galectin-2 is also a binding and regulatory partner of lymphotoxin-α (LTA), a pro-inflammatory cytokine. LTA has multiple functions in regulating the immune system and may contribute to inflammatory process leading to myocardial infarction (MI), coronary heart disease (CHD), and coronary artery disease (CAD). The regulatory mechanism of LTA secretion could be that galectin induces T cell apoptosis and thus effects cytokine secretion. Ozaki et al. found the genetic substitution affects the transcriptional level of galectin-2 in vitro, potentially resulting in altered secretion of LTA, thus affecting the degree of inflammation. The results indicated LTA and galectin-2 may have roles in the pathogenesis of MI. Also in the CHD study, the authors concluded that an association between LTA and galectin-2 gene polymorphisms and markers of inflammation and cell adhesion molecules was evident. Although significant association between LTA gene polymorphisms and CHD in American women and men had not been found, galectin-2 gene variant was significantly associated with a decreased risk of CHD.

Functional single nucleotide polymorphisms (SNP) studies suggest mutation in the galectin-2 gene in a Japanese population is associated with increased MI incidence. It is possible this is due to reduced galectin-2/LTA function causing increased inflammation leading to MI. However, Mangino et al attempted to replicate this in a British population and found no significant association between functional SNP (rs7291467) in galectin-2 and increased MI incidence.

However, most lectins require the presence of high concentrations of exogenous thiol reagents, DTT or mercaptoethanol, to maintain their carbohydrate-binding activity due to the existing of free cysteine residues (40). Chemical modification, such as using iodoacetamide (IDA) to block cysteine, has been utilized during galectin purification to stabilize carbohydrate binding activity (25,41). In this study, hGal2 WT was modified by site-directed mutagenesis. Sequencing and LC-MS results demonstrated the success of mutagenesis. Mutation C57M increased the expression solubility of hGal2 and also the storage stability. The improved stability may result from site-directed elimination of a cysteine residue, which has previously protected galectin-1 against random formation of disulfide bonds and the destruction of native structure (6). As a protein with great clinical potential, the successful site-directed mutagenesis of hGal2 resulting in improved expression level and stability will greatly simplify the industrial production and clinical application of this protein. Mutation of hGal2 to yield a variant having a single cysteine also opens the opportunity for the further specific chemical conjugation of a polymer, specifically monoPEGylation.

A key challenge during PEGylation is the need to develop reproducible and well-characterised production methods. The position of attachment and the number of PEG adducts are key concerns for PEGylation control. In this study, homogeneous monoPEGylated hGal2 C57M was obtained at high purity. Jeppesen et al. (42) reported, assuming a random coil configuration for PEG, that the equivalent spherical diameters of 2 kDa, 5 kDa, and 20 kDa PEG were around 35, 70, and 153 Å, respectively. Our gel-filtration chromatography results (FIG. 8A) demonstrate a size increase as a result of PEGylation. Bioanalyzer® 2100 analysis (FIG. 8B) also reflected the large exclusion volume of PEG. More importantly, Bioanalyzer® results suggested that each molecule of hGal2 dimer had been singly PEGylated. The final PEGylation product of hGal2 should exist as PEG-Gal-Gal-PEG in the native state. MALDI-MS (FIG. 8C) results further confirmed the correct molecular weight, which directly proved the well-controlled PEGylation procedure based on site-directed mutagenesis of hGal2.

One of the benefits of PEGylation is that the large exclusion volume of PEG alters the properties of bound molecules and results in a reduced clearance rate through the kidney and a prolonged half-life in serum (43,44). However, excessive PEGylation could also induce a decrease/loss of the bioactivity of modified proteins as a result of steric interference. The overall biological functions of PEG thus depend on the balance of these factors. Katre et al. (43) PEGylated recombinant interleukin 2 (IL-2) and found an almost 50% reduction in in vitro protein bioactivity after extensive chemical modification. However, in vivo, improved solubility and prolonged circulatory half-life time increased the activity 20-100 fold. In our study, the secondary structure of hGal2 C57M before and after PEGylation was investigated and compared with hGal2 WT using CD. As shown in FIG. 9, there was no significant difference between those spectra, suggesting that attachment of PEG did not significantly perturb hGal2 secondary structure. The binding activity of PEGylated hGal2 C57M was also explored on ASF-immobilized surface using SPR. As shown in FIG. 11D, an obvious reflectivity change was observed with PEGylated hGal2 C57M loading at low concentration. The results suggest that PEGylation modified but did not eliminate the binding activity of hGal2 to ASF.

This study successfully improved the expression solubility and storage stability of hGal2 by mutagenesis. More importantly, the mutation of hGal2 ensures well-controlled site-directed monoPEGylation, which did not cause significant hGal2 secondary structure change and did not eliminate ASF-binding activity. The success of site directed PEGylation is anticipated to extend hGal2's in vivo circulation life time and reduce the immunogenicity, antigenicity or toxicity of hGal2. Further bioactivity characterization in appropriate biological models is required to confirm the efficacy of this new monoPEGylated hGal2.

### Summary

Galectin-2 is a member of the beta-galactose-binding lectin family. It is primarily expressed in intestinal epithelial cells, functions as an inducer of T cell apoptosis, and shows therapeutic effects in a murine colitis model by down-regulating intestinal inflammation, suggesting clinical potential. In this work, single cysteine mutants of human galectin-2 (hGal2) were engineered in order to improve stability against in vitro aggregation and to provide a single conjugation site for PEGylation. The present inventors chose C57 as the mutation site, leaving another cysteine residue C75 as the conjugation site. Three mutants (C57M, C57A and C57S) were expressed in E. coli and product solubility compared; C57M was soluble while C57A and C57S formed inclusion bodies using the particular bacterial expression conditions and parameters described. However, it is contemplated and conceived that the solubility of C57A and C57S may be improved by varying expression conditions such as, but not limited to, growth temperature and incubation times. C57M demonstrated enhanced stability to in vitro aggregation compared with wild type, and did not require the addition of reducing agent to the formulation buffer to prevent aggregation during storage over 3 weeks. Site-directed PEGylation of C57M was performed via the C75 residue using PEG-5000 with a maleimide functional group, and highly homogenous conjugates (purity>97%) were obtained after ion-exchange chromatography. Mass spectroscopy, gel-filtration and SDS-PAGE analysis proved that each monomer of the hGal2 dimer was conjugated with one PEG molecule. No secondary structure alteration upon PEGylation was observed by circular-dichroism spectroscopy, and surface plasmon resonance analysis showed that PEGylated C57M maintains an asialofetuin binding capability. The C57M mutant, together with its PEG conjugate, could be potentially useful for clinical application, with enhanced stability proved here and longer in vivo circulation life time anticipated following PEGylation.

### Example 2

Testing of Therapeutic Efficacy of Mutant and/or Derivative Forms of Galectin-2

The potential therapeutic efficacy of the mutant and/or derivative forms of galectin-2 of the present invention will be tested in experimental colitis in an animal model. Acute and chronic colitis will be induced in mice, and in particular BALB/c mice, via administration of dextran sodium sulphate (DSS) in drinking water. Negative control mice will be treated with a control solution such as isotonic sterile saline. A positive therapeutic control will be included where DSS-induced mice will be treated with a known therapeutic agent effective against colitis. The therapeutic potential of mutant and/or derivatives of galectin-2 will be tested by an administration regime comprising intraperitoneal injection of various doses of galectin-2 mutants and/or derivatives in the range of 0.01 to 5 mg/kg bodyweight. The galectin-2 proteins will be in a purified form suitable for injection into an animal. The time course of the experiment and number of doses will be dependent on whether an acute or chronic colitis model is being evaluated.

The effect of early administration of galectin-2 mutants and/or derivatives on acute colitis will be monitored by the Rachmilewitz disease activity index as described in Paclick et al 2008. Histological evidence of inflammation on colonic sections will also be determined.

The therapeutic ability of galectin-2 mutants and/or derivatives to modulate chronic intestinal inflammation will also be evaluated using the Rachmilewitz disease activity index and histological injury.

Cytokine profiles of lamina propria mononuclear cells will also be monitored in the treated acute and chronic experimental colitis models as well as the effect of galectin-2 mutants and/or derivatives on apoptosis of lamina propria cells.

The protective efficacy of galectin-2 mutants and/or derivatives of the present invention will also be tested using T cell-mediated model of transfer colitis in immune mice (Holzlohner et al, 2007, Gastroenterology 132: A571). In this model, intestinal inflammation will be induced followed by treatment with galectin-2 mutants and/or derivatives of the present invention.

### Example 3

Testing of LTA-Binding Ability of Mutant and/or Derivative Forms of Galectin-2

Wild-type galectin 2 interacts with LTA as shown by Ozaki et al, 2004, Nature, 429: 72. The LTA-binding ability of the mutant and/or derivative forms of galectin-2 of the present invention will be tested by a combination of methods. Ozaki et al, 2004 provides non-limiting examples of such methods.

An in-vitro binding assay will be performed using recombinant LTA and galectin-2 mutant and/or derivative forms. The recombinant proteins will be tagged with an affinity tag such as FLAG or a histidine tag. LTA and galectin-2 mutant and/or derivative will be combined and their direct binding confirmed by an in-vitro binding assay using monoclonal or polyclonal antibodies to LTA and galectin-2.

Co-immunoprecipitation of tagged LTA and galectin-2 will be performed in mammalian cells such as COST or HeLa cells. Immunoprecipitation will be performed using an antibody directed against the affinity tag or an anti-LTA or galectin-2 antibody. The immune complex will be visualised using a suitable method such as autoradiography or enzyme-based methods.

Subcellular co-localisation of LTA and galectin-2 mutants and/or derivatives in mammalian cells will also be monitored by immunohistochemistry.

SPR as hereinbefore described will also be utilised to examine the interaction between LTA and galectin-2 mutants and/or derivatives. An E. coli two hybrid system will also be used to investigate the interaction between LTA and galectin-2 mutants and/or derivatives.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

1. Gitt, M. A., Massa, S. M., Leffler, H., and Barondes, S. H. (1992) Journal of Biological Chemistry 267(15), 10601-10606
2. Lahm, H., Andre, S., Hoeflich, A., Fischer, J. R., Sordat, B., Kaltner, H., Wolf, E., and Gabius, H.-J. (2001) Journal of Cancer Research and Clinical Oncology 127(6), 375-386
3. Ozaki, K., Inoue, K., Sato, H., Iida, A., Ohnishi, Y., Sekine, A., Sato, H., Odashiro, K., Nobuyoshi, M., Hori, M., Nakamura, Y., and Tanaka, T. (2004) Nature 429(6987), 72-75
4. Sturm, A., Lensch, M., Andre, S., Kaltner, H., Wiedenmann, B., Rosewicz, S., Dignass, A. U., and Gabius, H.-J. (2004) Journal of Immunology 173(6), 3825-3837
5. Sturm, A., Paclik, D., Wiedenmann, B., Gabius, H.-J., and Dignass, A. U. (2006) Inflammatory Bowel Diseases 12, S28-S28
6. Hirabayashi, J., and Kasai, K. (1991) Journal of Biological Chemistry 266(35), 23648-23653
7. Harris, J. M. (1992) Poly(ethylene glycol): chemistry and biological applications., Plenum Press, New York
8. Harris, J. M., Martin, N. E., and Modi, M. (2001) Clinical Pharmacokinetics 40(7), 539-551
9. Veronese, F. M., and Harris, J. M. (2002) Advanced Drug Delivery Reviews 54(4), 453-456
10. Nucci, M. L., Shorr, R., and Abuchowski, A. (1991) Advanced Drug Delivery Reviews 6(2), 133-151
11. Greenwald, R. B., Choe, Y. H., McGuire, J., and Conover, C. D. (2003) Advanced Drug Delivery Reviews 55(2), 217-250
12. Shen, L. J., and Shen, W. C. (2006) Current Opinion in Molecular Therapeutics 8(3), 240-248
13. Veronese, F. M., and Pasut, G. (2005) Drug Discovery Today 10(21), 1451-1458
14. Mohanty, S. R., Kupfer, S. S., and Khiani, V. (2006) Nature Clinical Practice Gastroenterology & Hepatology 3(8), 446-458
15. Roberts, M. J., Bentley, M. D., and Harris, J. M. (2002) Advanced Drug Delivery Reviews 54(4), 459-476
16. Zalipsky, S. (1995) Advanced Drug Delivery Reviews 16(2-3), 157-182
17. Lobsanov, Y. D., Gitt, M. A., Leffler, H., Barondes, S. H., and Rini, J. M. (1993) Journal of Biological Chemistry 268(36), 27034-27038
18. Ruan, H., Lunnen, K. D., Pelletier, J. J., and Xu, S. Y. (1997) Gene 188(1), 35-39
19. Zaccolo, M., Williams, D. M., Brown, D. M., and Gherardi, E. (1996) Journal of Molecular Biology 255(4), 589-603
20. Chapman, A. P., Antoniw, P., Spitali, M., West, S., Stephens, S., and King, D. J. (1999) Nature Biotechnology 17(8), 780-783
21. Tsutsumi, Y., Onda, M., Nagata, S., Lee, B., Kreitman, R. J., and Pastan, I. (2000) Proceedings of the National Academy of Sciences of the United States of America 97(15), 8548-8553
22. Yamamoto, Y., Tsutsumi, Y., Yoshioka, Y., Nishibata, T., Kobayashi, K., Okamoto, T., Mukai, Y., Shimizu, T., Nakagawa, S., Nagata, S., and Mayumi, T. (2003) Nature Biotechnology 21(5), 546-552
23. Sato, H. (2002) Advanced Drug Delivery Reviews 54(4), 487-504
24. DeFrees, S., Wang, Z. G., Xing, R., Scott, A. E., Wang, J., Zopf, D., Gouty, D. L., Sjoberg, E. R., Panneerselvam, K., Brinkman-Van Der Linden, E. C. M., Bayer, R. J., Tarp, M. A., and Clausen, H. (2006) Glycobiology 16(9), 833-843
25. Gabius, H.-J. (1990) Analytical Biochemistry 189(1), 91-94
26. Knoll, W. (1998) Annual Review of Physical Chemistry 49, 569-638
27. Knoll, W., Zizlsperger, M., Liebermann, T., Arnold, S., Badia, A., Liley, M., Piscevic, D., Schmitt, F. J., and Spinke, J. (2000) Colloids and Surfaces A 161(1), 115-137
28. Hirabayashi, J., Hashidate, T., Arata, Y., Nishi, N., Nakamura, T., Hirashima, M., Urashima, T., Oka, T., Futai, M., Muller, W. E. G., Yagi, F., and Kasai, K. (2002) Biochimica Et Biophysica Acta-General Subjects 1572(2-3), 232-254
29. Miura, T., Takahashi, M., Horie, H., Kurushima, H., Tsuchimoto, D., Sakumi, K., and Nakabeppu, Y. (2004) Cell Death and Differentiation 11(10), 1076-1083
30. Liao, D. I., Kapadia, G., Ahmed, H., Vasta, G. R., and Herzberg, O. (1994) Proceedings of the National Academy of Sciences of the United States of America 91(4), 1428-1432
31. Spiro, R. G. (1960) Journal of Biological Chemistry 235(10), 2860-2869
32. Green, E. D., Adelt, G., Baenziger, J. U., Wilson, S., and Vanhalbeek, H. (1988) Journal of Biological Chemistry 263(34), 18253-18268
33. Nilsson, B., Norden, N. E., and Svensson, S. (1979) Journal of Biological Chemistry 254(11), 4545-4553
34. Dam, T. K., Gabius, H.-J., Andre, S., Kaltner, H., Lensch, M., and Brewer, C. F. (2005) Glycobiology 15(11), 1215-1215
35. Rubinstein, N., Ilarregui, J. M., Toscano, M. A., and Rabinovich, G. A. (2004) Tissue Antigens 64(1), 1-12
36. Almkvist, J., and Karlsson, A. (2002) Glycoconjugate Journal 19(7-9), 575-581
37. van den Brule, F., Califice, S., and Castronovo, V. (2002) Glycoconjugate Journal 19(7-9), 537-542
38. Hughes, R. C. (2002) Glycoconjugate Journal 19(7-9), 621-629
39. Watt, D. J., Jones, G. E., and Goldring, K. (2004) Glycoconjugate Journal 19(7-9), 615-619
40. Barondes, S. H. (1984) Science 223(4642), 1259-1264
41. Powell, J. T., and Whitney. P. L. (1984) Biochemical Journal 223(3), 769-774
42. Jeppesen, C., Wong, J. Y., Kuhl, T. L., Israelachvili, J. N., Mullah, N., Zalipsky, S., and Marques, C. M. (2001) Science 293(5529), 465-468
43. Katre, N. V., Knauf, M. J., and Laird, W. J. (1987) Proceedings of the National Academy of Sciences of the United States of America 84(6), 1487-1491
44. Knauf et al (1988) Journal of Biological Chemistry 263(29), 15064-1507

1. An isolated modified galectin-2 protein selected from the group consisting of an isolated galectin-2 protein comprising a mutation of cysteine 57 or an analogous residue, an isolated galectin-2 protein comprising a modification of cysteine 75 or analogous residue and an isolated galectin-2 protein comprising a mutation of cysteine 57 and a modification of cysteine 75 or analogous residues, with respect to a wild-type galectin-2 amino acid sequence.

2. The isolated modified galectin-2 protein according to claim 1, wherein the mutation improves solubility and/or stability of said isolated modified galectin-2 protein relative to a wild-type galectin-2 protein.

3. The isolated modified galectin-2 protein according to claim 2, wherein the mutation is a substitution selected from a conservative substitution and a non-conservative substitution.

4. The isolated modified galectin-2 protein according to claim 2, wherein the cysteine 57 is substituted with an amino acid residue selected from the group consisting of a methionine residue, an alanine residue and a serine residue.

5-6. (canceled)

7. The isolated modified galectin-2 protein according to claim 1, wherein the modification improves pharmacokinetics and/or pharmacodynamics of said isolated modified galectin-2 protein relative to a galectin-2 protein which has not been modified at cysteine 75.

8. The isolated modified galectin-2 protein according to claim 1, wherein the modification is a chemical modification selected from treatment with an alkylating agent and attachment of one or more polymer molecules.

9. The isolated modified galectin-2 protein according to claim 8, wherein the or each polymer molecule is selected from a polyethylene glycol (PEG) and a dextran.

10-19. (canceled)

20. An isolated nucleic acid which encodes an isolated modified galectin-2 protein according to any one of the preceding claims according to claim 1.

21. The isolated nucleic acid according claim 20, which is DNA.

22. (canceled)

23. A genetic construct comprising an isolated nucleic acid according to claim 20 operably-linked to one or more regulatory nucleotide sequences in a vector.

24. The genetic construct according to claim 23, which is an expression construct.

25-26. (canceled)

27. A host cell comprising a genetic construct according to claim 23.

28. (canceled)

29. An isolated antibody which binds to and/or has been raised against an isolated modified galectin-2 protein according to claim 1, wherein said isolated antibody does not bind wild-type galectin-2 protein or binds to a wild-type galectin-2 protein with relatively lower affinity.

30. A method of producing an isolated modified galectin-2 protein for use in a pharmaceutical composition, said method including the steps of:
mutating an isolated galectin-2 protein at cysteine 57 or an analogous residue, with respect to a wild-type galectin-2 amino acid sequence; and/or
(ii) modifying an isolated galectin-2 protein at cysteine 75 or an analogous residue, with respect to a wild-type galectin-2 amino acid sequence; or
(iii) modifying the mutated galectin-2 protein of step (i), at cysteine 75 or an analogous residue, with respect to a wild-type galectin-2 amino acid sequence,
to thereby produce the isolated modified galectin-2 protein for use in a pharmaceutical composition.

31. (canceled)

32. The method of producing an isolated modified galectin-2 protein for use in a pharmaceutical composition according to claim 30, wherein the mutation is a substitution selected from a conservative substitution and a non-conservative substitution.

33-35. (canceled)

36. The method of producing an isolated modified galectin-2 protein for use in a pharmaceutical composition according to claim 30, wherein the modification is a chemical modification selected from treatment with an alkylating agent and attachment of one or more polymer molecules.

37. The method of producing an isolated modified galectin-2 protein for use in a pharmaceutical composition according to claim 36, wherein the or each polymer molecule is selected from a polyethylene glycol (PEG) and a dextran.

38-45. (canceled)

46. An isolated modified galectin-2 protein for use in a pharmaceutical composition produced according to a method of producing an isolated modified galectin-2 protein according to claim 30.

47. A pharmaceutical composition comprising an isolated modified galectin-2 protein according to claim 1, an isolated modified galectin-2 protein according to claim 46, a genetic construct according to claim 23 and/or an isolated nucleic acid according to claim 20, together with a pharmaceutically-acceptable carrier, diluent or excipient.

48. A method of modulating an immune response in an animal, said method including the step of administering an effective amount of a pharmaceutical composition according to claim 47, to thereby modulate the immune response in said animal.

49-57. (canceled)

58. A method of treating an animal, said method including the step of administering an effective amount of a pharmaceutical composition according to claim 47 to said animal to thereby modulate an immune response in said animal to prophylactically and/or therapeutically treat an inflammatory disease or a disease, disorder or condition responsive to inhibition or suppression of T cell activation.

59-62. (canceled)
